(19) United States
(12) Patent Application Publication
(10) Pub. No.: US 2019/0030077 A1
(43) Pub. Date: Jan. 31, 2019

(54) ENHANCEMENT OF STEM CELL ENGRAFTMENT WITH ONCOSTATIN M

(71) Applicants: President and Fellows of Harvard College, Cambridge, MA (US); The General Hospital Corporation, Boston, MA (US)

(72) Inventors: David T. Scadden, Weston, MA (US); Jonathan Hoggatt, Somerville, MA (US)

(21) Appl. No.: 15/773,983

(22) PCT Filed: Nov. 7, 2016
(86) PCT No.: PCT/US16/60829

§ 371 Date: May 4, 2018

Related U.S. Application Data
(60) Provisional application No. 62/251,633, filed on Nov. 5, 2015.

(51) Int. Cl. A61K 35/28; A61K 35/14; A61P 35/00

(52) U.S. Cl. CPC A61K 35/28; A61K 35/14; A61K 38/18; A61K 2300/00; A61P 35/00

(57) ABSTRACT

Disclosed herein novels methods and compositions that are useful for enhancing stem cell homing to, and engraftment in the target, tissues of a subject following stem cell transplant. In certain aspects, the inventions disclosed herein comprise a step of administering oncostatin M or a biologically active fragment, mutant, analog or fusion construct thereof to the subject and thereby increasing the stem cell homing and engraftment efficiency to the target tissues of the subject. Such methods and compositions may be used to improve subject survival and outcomes following, for example, hematopoietic stem cell transplant.

### RELATED APPLICATIONS

This application claims the benefit of U.S. Provisional Application No. 62/251,633, filed Nov. 5, 2015, the entire teachings of which are incorporated herein by reference.

### GOVERNMENT SUPPORT

This invention was made with government support under HL119559 awarded by the National Institutes of Health. The government has certain rights in the invention.

### BACKGROUND OF THE INVENTION

Hematopoietic stem cell (HSC) transplantation is a common life-saving medical procedure used to treat and cure approximately 60,000 patients per year globally, and each year over 18,000 bone marrow transplants are performed in the United States. HSCs reside within a microenvironmental stem cell niche, the cellular makeup of which is complex, with contributions from endothelial, mesenchymal, and mature hematopoietic cells. Despite its common use, for example, to treat hematologic, metabolic and malignant diseases, there remain critical unmet needs to improve transplant efficiency and to enhance hematopoietic engraftment. There also remains a need to identify new compositions and methods of enhancing hematopoietic engraftment.

### SUMMARY OF THE INVENTION

Hematopoietic stem cells (HSCs) reside in specific niches that control survival, proliferation, self-renewal or differentiation in the bone marrow. The trafficking of HSCs between the bone marrow compartment and the blood in a normal subject may fill empty or damaged niches, as well as contribute to the maintenance of normal hematopoiesis. The inventions described herein relate to G-CSF-mediated and macrophage-mediated mobilization of HSCs, and thus provide novel therapeutic methods that are useful for enhancing HSC and progenitor stem cell homing to certain tissues, such as the bone marrow compartment and the stem cell niche.

The inventions disclosed herein contribute to the realization of the full potential of HSC and progenitor stem cell transplantation, and disclose the factors that contribute to stem cell retention in the bone marrow stem cell niche, as well as new therapeutics that can be administered to a transplant recipient to enhance HSC and progenitor stem cell engraftment. In particular, the methods and therapeutics disclosed herein may be used to enhance homing and engraftment of HSCs to a subject's bone marrow stem cell niche, in addition to having other desirable characteristics.

Accordingly, disclosed herein are methods and compositions for enhancing homing and engraftment of transplanted stem cells to a target tissue of a subject. In certain aspects, such methods and compositions relate to or comprise the identification of factors that promote HSC and progenitor stem cell retention in one or more target tissues of a subject (e.g., the bone marrow stem cell niche). The methods and compositions disclosed herein are useful for promoting HSC retention in one or more target tissues, as well as enhancing the engraftment efficiency of transplanted stem cells (e.g., HSCs) in a target tissue of a subject.

In certain embodiments, disclosed herein are methods of increasing stem cell engraftment efficiency in a target tissue of a subject, the method comprising administering oncostatin M (e.g., human oncostatin M encoded by SEQ ID NO: 1) or a biologically active fragment, mutant, analog or fusion construct thereof to the subject (e.g., a subject in need of a stem cell transplant) and thereby increasing the stem cell engraftment efficiency in the target tissues (e.g., bone marrow tissues) of the subject.

In some aspects, disclosed herein are methods of increasing stem cell homing to a target tissue of a subject (e.g., a human subject), the method comprising administering oncostatin M (e.g., human oncostatin M encoded by SEQ ID NO: 1) or a biologically active fragment, mutant, analog or fusion construct thereof to the subject and thereby increasing the stem cell homing to the target tissue of the subject.

In certain embodiments, also disclosed herein are methods of reducing the mobilization of stem cells from a target tissue of a subject, the method comprising administering oncostatin M (e.g., human oncostatin M encoded by SEQ ID NO: 1) or a biologically active fragment, mutant, analog or fusion construct thereof to the subject and thereby reducing mobilization of the stem cells from the target tissue of the subject.

In still other embodiments, disclosed herein are methods of increasing stem cell homing to a target tissue of a subject, the method comprising: (a) administering oncostatin M (e.g., human oncostatin M encoded by SEQ ID NO: 1) or a biologically active fragment, mutant, analog or fusion construct thereof to the subject; and (b) administering the stem cells to the subject, wherein the stem cell homing to the target tissue is increased as compared to a method performed using only the step of administering the stem cells to the subject. For example, the stem cells homing to the target tissue may be increased by at least about 5%, 10%, 12.5%, 15%, 17.5%, 20%, 25%, 30%, 35%, 40%, 45%, 50%, 55%, 60%, 65%, 70%, 75%, 80%, 85%, 90%, 95%, 97.5%, 99%, 100%, 110%, 125%, 150%, 200%, 300%, 400%, 500% or more relative to a method performed using only the step of administering the stem cells to the subject.

In some embodiments, the inventions disclosed herein are directed to methods of increasing stem cell retention in a target tissue of subject, the method comprising: (a) administering oncostatin M (e.g., human oncostatin M encoded by SEQ ID NO: 1) or a biologically active fragment, mutant, analog or fusion construct thereof to the subject; and (b) administering the stem cells to the subject, wherein the retention of the stem cells in the target tissue is increased as compared to a method performed using only the step of administering the stem cells to the subject. For example, the retention of the stem cells in the target tissue may be increased by at least about 5%, 10%, 12.5%, 15%, 17.5%, 20%, 25%, 30%, 35%, 40%, 45%, 50%, 55%, 60%, 65%, 70%, 75%, 80%, 85%, 90%, 95%, 97.5%, 99%, 100%, 110%, 125%, 150%, 200%, 300%, 400%, 500% or more relative to a method performed using only the step of administering the stem cells to the subject.

In yet other embodiments, provided herein are methods of increasing stem cell engraftment efficiency in a target tissue of a subject, the method comprising: (a) administering oncostatin M (e.g., human oncostatin M encoded by SEQ ID NO: 1) or a biologically active fragment, mutant, analog or fusion construct thereof to the subject; and (b) administering the stem cells to the subject, wherein the stem cell engraftment efficiency is increased as compared to a method performed using only the step of administering the stem cells to the subject. For example, the stem cell engraftment efficiency in the target tissue may be increased by at least about 5%, 10%, 12.5%, 15%, 17.5%, 20%, 25%, 30%, 35%, 40%, 45%, 50%, 55%, 60%, 65%, 70%, 75%, 80%, 85%, 90%, 95%, 97.5%, 99%, 100%, 110%, 125%, 150%, 200%, 300%, 400%, 500% or more relative to a method performed using only the step of administering the stem cells to the subject.

In certain aspects, disclosed herein are methods of engrafting stem cells in a target tissue of a subject, the method comprising: (a) administering oncostatin M or a biologically active fragment, mutant, analog or fusion construct thereof to the subject; and (b) administering the stem cells to the subject, thereby engrafting the stem cells in the target tissue of the subject.

In some embodiments of the present invention, the subject is pre-treated with the oncostatin M or a biologically active fragment, mutant, analog or fusion construct thereof prior to administering or transplanting stem cells to the subject. For example, oncostatin M or a biologically active fragment, mutant, analog or fusion construct thereof may be administered to the subject for at least about two days prior to administering stem cells to the subject. In some embodiments, oncostatin M or a biologically active fragment, mutant, analog or fusion construct thereof is administered about every six hours for about two days prior to administering stem cells to the subject. In certain aspects, oncostatin M or a biologically active fragment, mutant, analog or fusion construct thereof is administered to the subject after the target tissues of the subject (e.g., bone marrow tissues) have been conditioned for stem cell engraftment (e.g., conditioned using irradiation or other myeloablative conditioning). For example, a subject may receive myeloablative conditioning prior to the step of administering oncostatin M or a biologically active fragment, mutant, analog or fusion construct thereof to the subject.

The methods and compositions disclosed herein advantageously enhance the engraftment efficiency of the transplanted stem cells (e.g., HSCs and/or progenitor cells). In certain aspects, such methods and compositions enhance or otherwise increase the stem cell engraftment efficiency by at least about two-fold (e.g., by at least about two-, three-, four-, five-, six-, seven-, eight, nine-, ten-fold or more). In certain aspects, such methods and compositions enhance or otherwise increase the stem cell engraftment efficiency by at least about three-fold. In yet other embodiments, such methods and compositions enhance or otherwise increase the stem cell engraftment efficiency by at least about four-fold.

In certain aspects, the methods and compositions disclosed herein enhance or otherwise increase stem cell (e.g., HSC and/or progenitor cells) homing to a target tissue (e.g., a target tissue such as the bone marrow stem cell niche). In some embodiments, such methods and compositions increase the stem cell homing by at least about two-fold (e.g., by at least about two-, three-, four-, five-, six-, seven-, eight, nine-, ten-fold or more). In certain aspects, such methods and compositions enhance or otherwise increase stem cell homing efficiency by at least about three-fold. In yet other embodiments, such methods and compositions enhance or otherwise increase stem cell homing efficiency by at least about four-fold.

The methods disclosed herein comprise a step of administering to a subject oncostatin M or a biologically active fragment, mutant, analog or fusion construct thereof. In certain aspects, the oncostatin M is recombinantly prepared. In certain aspects, the administered oncostatin M or a biologically active fragment, mutant, analog or fusion construct thereof stimulates production of stromal derived factor-1 (SDF-1) in the bone marrow stem cell niche of the subject. In certain embodiments disclosed herein, the oncostatin M is or comprises the human mature oncostatin M or a biologically active fragment, mutant, analog or fusion construct thereof. In certain embodiments, oncostatin M comprises SEQ ID NO: 1. In yet other embodiments, the oncostatin M comprises residues 26-220 of SEQ ID NO: 1, corresponding to the human mature oncostatin M. In still other embodiments, the oncostatin M comprises residues 1-220 of SEQ ID NO: 1. In some embodiments, oncostatin M comprises residues 220-252 of SEQ ID NO: 1, corresponding to the human oncostatin M propeptide.

The methods and compositions disclosed are useful for enhancing or otherwise increasing stem cell engraftment efficiency or stem cell homing efficiency in one or more target tissues of a subject. In certain aspects, the target tissue comprises the subject's stem cell niche. In certain aspects, the target tissue comprises a subject's bone marrow stem cell niche.

The methods and compositions disclosed herein may be useful for increasing the engraftment efficiency or homing efficiency of stem cells in one or more target tissues of the subject. In certain aspects, the stem cells comprise hematopoietic stem cells (HSCs). In some embodiments, the stem cells comprise progenitor cells. In yet other embodiments, the stem cells comprise hematopoietic progenitor cells. In certain embodiments, the stem cells comprise exogenous stem cells. Alternatively, in other embodiments the stem cells comprise the subject's endogenous stem cells (e.g., a subject's endogenous HSCs or progenitor stem cell that are genetically modified or that have been genetically corrected).

In certain aspects, the methods disclosed herein enhance retention and engraftment of the stem cells in the target tissue of the subject. For example, in certain aspects, a subject may be administered oncostatin M or a biologically active fragment, mutant, analog or fusion construct thereof to enhance retention of transplanted HSCs and progenitor stem cells in the target tissues (e.g., the bone marrow stem cell niche) of a subject.

In certain embodiments, the methods and compositions disclosed herein increase the rate of subject survival from a stem cell disorder (e.g., a hematologic malignancy), as compared to a method performed without the step of administering oncostatin M or a biologically active fragment, mutant, analog or fusion construct thereof to the subject. For example, in certain aspects, the methods and compositions disclosed herein increase the rate of subject survival by at least about 100% (e.g., by at least about 100%. 110%, 120%, 125%, 150%, 200%, 250%, 300%, 400%, 500% or more).

While certain embodiments of the present inventions contemplate administering OSM or a biologically active fragment or variant thereof to a subject, it should be understood that the inventions are not intended to be limited to such embodiments. Rather, also contemplated are any compositions or methods capable of causing an increase in the concentration of OSM in the subject or in the subject's tissues. Accordingly, also disclosed herein are methods of increasing stem cell engraftment efficiency in a target tissue of a subject, such method comprising a step of increasing OSM in the subject and thereby increasing the stem cell engraftment efficiency in the target tissue of the subject.

In certain aspects, also disclosed are methods of increasing homing to or retention of stem cells in a target tissue of a subject, such methods comprising increasing oncostatin M in the subject (e.g., increasing the concentration or quantity of OSM) and thereby increasing the homing to or retention of the stem cells in the target tissue of the subject.

In some embodiments, also disclosed herein are methods of engrafting stem cells in a target tissue of a subject, such method comprising the steps of: (a) increasing OSM in the subject (e.g., increasing the concentration or quantity of OSM); and (b) administering the stem cells to the subject, thereby engrafting the stem cells in the target tissue of the subject.

In certain aspects, OSM is increased in the subject by administering to the subject OSM or a biologically active fragment, mutant, analog or fusion construct thereof. For example, in certain embodiments, OSM comprises SEQ ID NO: 1 or a biologically active fragment or variant thereof. In some embodiments, the OSM comprises residues 26-220 of SEQ ID NO: 1 or a biologically active fragment or variant thereof.

In some embodiments, the quantity or concentration of OSM in a subject's tissues (e.g., in the bone marrow compartment) may be increased by increasing the subject's endogenous production of oncostatin M. For example OSM may be increased by administering one or more cells stably expressing OSM to the subject. In some embodiments, such cells comprise the subject's endogenous cells. In some embodiments, such endogenous cells have been transformed such that they stably express OSM. In yet other embodiments, such endogenous cells have been transformed using an expression vector.

In some embodiments, the concentration or quantity of OSM in the tissues of a subject may be increased by increasing the concentration of OSM expressing cells in the subject. For example, in certain aspects OSM may be increased by increasing the population macrophage in the target tissue, thereby increasing OSM in the subject.

The methods and compositions disclosed herein are particularly useful in the treatment of diseases affecting a subject. In certain aspects, the methods and compositions disclosed herein are useful for the treatment of a subject in need of treatment for a hematologic or oncologic disease. For example, such methods and compositions are useful for treating a subject that has, is in need of treatment for, or is otherwise affected by a hematologic or oncologic disease selected from the group consisting of leukemia, lymphoma, multiple myeloma and other plasma cell dyscrasias (PCD), and myelodysplastic syndrome. In certain aspects, the subject is in need of a bone marrow transplant. In certain aspects, the subject is a mammal. In certain aspects, the subject is a human.

In some embodiments, the subject has or is affected by a hematological malignancy. For example, the subject may be in need of treatment for a hematological malignancy selected from the group consisting of acute lymphoid leukemia, acute myeloid leukemia, chronic lymphoid leukemia, chronic myeloid leukemia, myelo-proliferative disorders (MPD), Hodgkin's disease (HD), diffuse large B-cell non-Hodgkin's lymphoma, mantle cell lymphoma, lymphoblastic lymphoma, Burkitt's lymphoma, follicular B-cell non-Hodgkin's lymphoma, T-cell non-Hodgkin's lymphoma, lymphocyte predominant nodular Hodgkin's lymphoma, multiple myeloma and other plasma cell dyscrasias (PCD), and juvenile myelomonocytic leukemia.

In certain embodiments, the subject has or is affected by a non-malignant disease. For example, the subject may be in need of treatment for a non-malignant disease selected from the group consisting of myelofibrosis, myelodysplastic syndrome, amyloidosis, severe aplastic anemia, paroxysmal nocturnal hemoglobinuria, immune cytopenias, systemic sclerosis, rheumatoid arthritis, multiple sclerosis, systemic lupus erythematosus, Crohn's disease, chronic inflammatory demyelinating polyradiculoneuropathy, human immunodeficiency virus (HIV), Fanconi anemia, sickle cell disease, beta thalassemia major, Hurler's syndrome (MPS-IH), adrenoleukodystrophy, metachromatic leukodystrophy, familial erythrophagocytic lymphohistiocytosis and other histiocytic disorders, severe combined immunodeficiency (SCID), and Wiskott-Aldrich syndrome.

In certain aspects, the methods and compositions disclosed herein are practiced or otherwise administered to a subject prior to, in combination with, or following the administration to the subject of a therapeutically effective amount of a conventional treatment for a hematologic disease, oncologic disease, hematologic malignancy or non-malignant disease.

Oncostatin M or a biologically active fragment, mutant, analog or fusion construct thereof may be formulated for administration to a subject (e.g., a mammal) using any suitable routes of administration (e.g., orally, parenterally, intramuscularly, subcutaneously, topically, nasally or transdermally). In certain embodiments, oncostatin M or a biologically active fragment, mutant, analog or fusion construct thereof is administered to the subject parenterally. In some embodiments, oncostatin M or a biologically active fragment, mutant, analog or fusion construct thereof is formulated with one or more pharmaceutically acceptable carriers.

Similarly, oncostatin M or a biologically active fragment, mutant, analog or fusion construct thereof may be prepared or reconstituted in any suitable dosage form (e.g., a tablet, a capsule, a solution, a suspension, an ointment, a gel or a cream.) In certain aspects, oncostatin M or a biologically active fragment, mutant, analog or fusion construct thereof is formulated with one or more pharmaceutically acceptable carriers. In certain embodiments, oncostatin M or a biologically active fragment, mutant, analog or fusion construct thereof may be formulated for extended- or delayed-release or may be prepared in a glycosylated or pegylated form.

The above discussed, and many other features and attendant advantages of the present inventions will become better understood by reference to the following detailed description of the invention.

### BRIEF DESCRIPTION OF THE DRAWINGS

The patent or application file, which includes the appendix attached hereto, contains at least one drawing executed in color. Copies of this patent or patent application publication with color drawings will be provided by the Office upon request and payment of the necessary fee.

FIG. 1 depicts the macrophage and MS-5 co-culture system used by the present inventors to determine the factors that macrophages may be secreting that supports the stem cell niche. In particular, FIG. 1 illustrates the setup of a co-culture system in which media containing secretions from macrophages was collected and then cultured together with SDF-1 producing stromal cells to see if SDF-1 production was stimulated by anything in the media.

FIG. 2 shows the results of the co-culture assay depicted in FIG. 1 and confirms that macrophage conditioned media stimulates SDF-1 production.

FIG. 3 illustrates that oncostatin M (OSM) stimulates SDF-1 production in vitro when cultured with stromal cells.

FIG. 4 demonstrates that OSM mitigates the negative regulatory effects of G-CSF on SDF-1, thereby suggesting that OSM is the key molecule secreted by macrophages to regulate hematopoietic stem cell (HSC) retention within the stem cell niche.

FIG. 5 illustrates that OSM rescues HSC mobilization after macrophage depletion in vivo. In particular, depicted are the results of an in vivo experiment in which HSC mobilization was induced by depletion of macrophages using liposomes loaded with clodronate, a toxin that kills macrophages. As illustrated, clodronate treatment led to an increase in SKLs (HSCs) in the blood, but co-treatment with OSM was able to rescue the mobilization.

FIG. 6 depicts an experimental protocol designed to mimic what happens in a clinical setting. As illustrated in FIG. 6, the mice were irradiated to mimic the treatment a subject might receive to deplete their blood system and, after 48 hours, during which mice received OSM treatment every 6 hours, the mice receive a transplant, subsequent to which further analysis specific to that experiment was performed.

FIG. 7 depicts an experimental protocol designed to determine whether treatment with OSM could improve homing of HCSs to targeted tissues. As illustrated, 20 mice were irradiated, treated with OSM or vehicle control, and then transplanted with an enriched population of GFP+ stem and progenitor cells. 14 hours after transplant, femurs were pulled and flushed to obtain bone marrow for flow cytometry analysis looking at how many GFP+ cells and GFP+ HSCs from the donor were present in or homed to in the bone marrow.

FIG. 8 depicts the results of the experiment illustrated in FIG. 7. As illustrated in FIG. 8, mice that were pre-treated with OSM prior to transplantation exhibited an approximately 2-fold increase in HSC homing relative to mice that were administered the PBS control (n=10 mice per group, three independent experiments, P<0.05), thereby evidencing that OSM significantly increased homing of stem and progenitor cells to target tissues.

FIG. 9 depicts an experimental protocol designed to determine whether treatment with oncostatin M leads to increased hematopoietic expansion post-transplant. Cohorts of mice were sacrificed at days 4, 7, 11, and 14, their bone marrow was plated in methylcellulose, and the number of colony forming units (CFUs) were determined as a measure of progenitor and stem cell expansion.

FIG. 10 illustrates the results of the experiment depicted in FIG. 9 and demonstrates that pre-treatment with OSM led to increased hematopoietic expansion post-transplant.

FIG. 11 depicts an experimental protocol designed to determine whether treatment with oncostatin M led transplanted HSCs to produce mature blood cells to repopulate the hematopoietic system of the recipient.

FIGS. 12A-12C depict the results of the experimental protocol illustrated in FIG. 11. As illustrated, the average neutrophil (FIG. 12A), platelet (FIG. 12B) and white blood cell (FIG. 12C) recoveries were comparable, and in many instances higher in the OSM pre-treated mice. These results therefore confirm that the transplanted HSCs produce mature blood cells to repopulate the hematopoietic system of the recipient mice (N=10 mice per group, per experiment, 2 independent experiments, total of N=20 mice per group).

FIG. 13 depicts an experimental protocol used to determine whether the observed expansion and repopulation leads to increased subject survival after transplant. After transplant, mice were observed for 30 days to determine the percent that survived the transplant procedure.

FIG. 14 illustrates the results of the experimental protocol depicted in FIG. 13 and evidences that OSM pre-treatment is associated with better survival after radiation and transplant, relative to control. In particular, FIG. 14 illustrates the enhanced hematopoietic engraftment and survival observed in OSM pre-treated mice, with 100% survival, compared to 50% survival observed in mice treated with vehicle control (n=20 per group, P<0.001).

FIG. 15 illustrates hematopoietic stem cell (HSC) trafficking between bone marrow and peripheral blood. Stem cell transplant is largely possible because hematopoietic stem cells have an innate ability to “home” back to the bone marrow from the bloodstream after being infused into a patient. The reverse of this process is termed “mobilization.” Various factors within the niche regulate this trafficking of HSCs. One key regulatory mechanism for HSC retention and homing within the BM is expression and production of SDF-1/CXCR4 by stromal cells.

FIG. 16 illustrates that macrophage regulate hematopoietic stem cell (HSC) retention within the bone marrow niche by producing a positive supporting factor in the niche. Macrophages also express the G-CSF receptor, and macrophage depletion mobilizes HSCs even in the absence of G-CSF. Macrophages also promote retention of HSCs in bone marrow, as measured by SDF-1 production.

FIG. 17 depicts the amino acid sequence of human oncostatin M (SEQ ID NO: 1). The highlighted residues 26-220 represent the mature OSM chain, while residues 1-25 represent the OSM signal peptide and residues 221-252 represent the OSM propeptide.

FIG. 18 depicts the nucleic acid sequence of human oncostatin M mRNA (SEQ ID NO: 2).

FIG. 19 depicts the amino acid sequence of human oncostatin M preprotein (SEQ ID NO: 3).

### DETAILED DESCRIPTION OF THE INVENTION

Oncostatin M is a 28 kDa multifunctional member of the IL-6 family of cytokines that is secreted by monocytes, macrophages, neutrophils and activated T-lymphocytes (Tanaka, et al., Rev Physiol Biochem Pharmacol 2003, 149: 39-533) and was originally isolated from conditioned media of a phorbol ester-treated histiocytic lymphoma cell line, U937, based on the ability to inhibit the growth or development of a human melanoma cell line. Work described herein provides a previously unknown key regulatory mechanism governing G-CSF- and macrophage-mediated mobilization of HSCs and thus a novel therapeutic strategy to enhance HSC homing to and engraftment in target tissues. Specifically, the inventions disclosed herein are based on the finding that the administration of oncostatin M (OSM) prior to transplantation, as described in more detail in the Examples resulted in a 2-fold increase in HSC homing to the bone marrow compartment. Furthermore, these homed progenitors demonstrated remarkably enhanced hematopoietic expansion. Excitingly, using a limited cell number transplant and mimicking the settings of single cord blood unit transplantation in adults or other settings of limited HSC number, work described herein also demonstrates enhanced hematopoietic engraftment and survival in OSM pre-treated mice, with 100% survival compared to 50% survival of mice treated with vehicle control.

Disclosed herein are therapeutics, compositions and methods that are useful for enhancing hematopoietic engraftment in a subject. In certain aspects, the compositions and methods disclosed herein generally relate to the use of OSM as a factor that may be used to promote stem cell (e.g., hematopoietic stem cells (HSCs) or progenitor stem cells) retention in the bone marrow stem cell niche of a subject and the engraftment of transplanted stem cells to a target tissue of a subject (e.g., the bone marrow stem cell niche). As used herein, the phrase “stem cell niche” refers to the complex milieu composed of cells and extracellular matrix, as well as the signaling molecules associated with each population of stem cells. The physical structure of the niche varies between organisms and between stem cell types, its composition ranging from a single cell or cell type to many cells of varying cell types.

Aspects of the invention also relate to the use of OSM to enhance stem and progenitor cell homing to the bone marrow, the expansion and repopulation of cells within the bone marrow, and/or survival of transplant recipients post-transplant. By pre-treating or administering OSM to a subject prior to stem cell transplant, the engraftment efficiency of such transplanted stem cells (e.g., HSCs and progenitor stem cell) may be enhanced or increased.

Generally, the methods and compositions disclosed herein are useful for enhancing engraftment of hematopoietic stem cells and/or progenitor cells in the stem cell niche of a target tissue (e.g., bone marrow tissue). As used herein, the terms “hematopoietic stem cell” and “HSC” refers to stem cells that can differentiate into the hematopoietic lineage and give rise to all blood cell types such as white blood cells and red blood cells, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells). “Stem cells” are defined by their ability to form multiple cell types (multipotency) and their ability to self-renew. Hematopoietic stem cells can be identified, for example by cell surface markers such as CD34−, CD133+, CD48−, CD150+, CD244−, cKit+, Sca1+, and lack of lineage markers (negative for B220, CD3, CD4, CD8, Mac1, Gr1, and Ter119, among others). Methods of identifying and analyzing hematopoietic stem cells has been reviewed by Challen et al. (see e.g., “Mouse Hematopoietic Stem Cell Identification and Analysis,” Cytometry A. 2009; 75(1):14-24, incorporated herein by reference in its entirety). The methods described herein contemplate any stem cell which would be useful for transplantation, including, but not limited to, peripheral blood stem cells, bone marrow stem cells, umbilical cord stem cells, genetically modified stem cells, etc.

As used herein, the terms “hematopoietic progenitor cells” encompasses pluripotent cells which are committed to the hematopoietic cell lineage, generally do not self-renew, and are capable of differentiating into several cell types of the hematopoietic system, such as granulocytes, monocytes, erythrocytes, megakaryocytes, B-cells and T-cells, including, but not limited to, short term hematopoietic stem cells (ST-HSCs), multi-potent progenitor cells (MPPs), common myeloid progenitor cells (CMPs), granulocyte-monocyte progenitor cells (GMPs), megakaryocyte-erythrocyte progenitor cells (MEPs), and committed lymphoid progenitor cells (CLPs). The presence of hematopoietic progenitor cells can be determined functionally as colony forming unit cells (CFU-Cs) in complete methylcellulose assays, or phenotypically through the detection of cell surface markers (e.g., CD45−, CD34+, Ter119−, CD16/32, CD127, cKit, Sca1) using assays known to those of skill in the art.

The methods and compositions disclosed herein are useful in connection with stem cell (e.g., HSC) transplants, as well as for the treatment of diseases for which a stem cell transplant may be indicated. In certain aspects, such methods and compositions promote stem cell homing to one or more target tissues. Similarly, in certain embodiments, such methods and compositions enhance the retention of transplanted stem cells, and the engraftment efficiency of such transplanted stem cells (e.g., HSCs) in a target tissue of a subject. Accordingly, the methods and compositions disclosed herein are characterized by their enhanced or improved engraftment efficiency.

As used herein, the phrase “engraftment efficiency” generally refers to the efficiency with which an administered or transplanted stem cell population (e.g., HSCs or progenitor cells) engrafts in a target tissue of the subject (e.g., the conditioned bone marrow tissue of the subject). For example, in certain embodiments, pre-treating or otherwise administering OSM to a subject prior to a HSC transplant increases the engraftment efficiency of such transplanted HSCs in the bone marrow stem cell niche of the subject. In certain embodiments, the methods and compositions disclosed herein increase engraftment efficiency by at least about 5%, 7.5%, 10%, 12.5%, 15%, 20%, 25%, 30%, 35%, 40%, 50%, 60%, 70%, 75%, 80%, 90%, 95%, 100% or more. In certain aspects, the determination of engraftment efficiency is assessed relative to the engraftment efficiency of a method in which the subject is treated or the transplant is performed without OSM pre-treatment, in accordance with the methods disclosed herein.

In certain embodiments, the inventions disclosed herein contemplate the administration of an effective amount of OSM, or a biologically active fragment, mutant, analog or fusion construct thereof, or an agent which enhances or increases the amount or activity of OSM or a biologically active fragment, mutant, analog or fusion construct, to the subject. Cloning of a cDNA for OSM showed that it encodes a 227 amino acid polypeptide which is structurally and functionally related to the family of hematopoietic and neurotrophic cytokines whose members include leukemia inhibitory factor (LIF), interleukin-6 (IL-6), interleukin-11 (IL-11), ciliary neurotrophic factor (CNTF), and cardiotrophin (Rose, et al., Proc. Natl. Acad. Sci. U.S.A., 1991, 88: 8641-8645). Proteolytic cleavage near the carboxy-terminus of the secreted OSM yields the fully active form of OSM, 209 amino acids length having two N-linked glycosylation sites. As used herein, the terms “oncostatin M” and “OSM” generally include oncostatin M, as well as any biologically active variants thereof. For example, in certain aspects, OSM comprises or consists of SEQ ID NO: 1, SEQ ID NO: 2, SEQ ID NO: 3 or a biologically active fragment, mutant, analog or fusion construct thereof. In certain embodiments, OSM comprises or consists of residues 26-220 of SEQ ID NO: 1, which represents the human mature OSM. Oncostatin M is expressed as a pre-propolypeptide containing a signal peptide (residues 1-25 of SEQ ID NO: 1). After cleavage of the signal peptide, there remains a polypeptide of approximately 227 residues, which is further processed by proteolytic cleavage to yield the human mature polypeptide (residues 26-220 of SEQ ID NO: 1). In certain embodiments, OSM is encoded by SEQ ID NO: 2 (NM_020530), which corresponds to human OSM mRNA (NM_020530). In certain embodiments, OSM comprises the human oncostatin M preproprotein encoded by SEQ ID NO: 3 (NP_065391).

In some embodiments, the OSM or a biologically active fragment, mutant, analog or fusion thereof is at least about 50%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% identical to SEQ ID NO: 1.

In certain aspects, the mature OSM may be subject to further cleavage or modification, which preferably does not negatively influence its biological activity. Fragments, mutants, analogs and fusion constructs of OSM are considered to be biologically active if they demonstrate similar biologic properties as oncostatin M, for example, the ability to improve the engraftment efficiency of transplanted HSCs. Various genetic modifications of recombinant OSM can be used to encode biologically active OSM molecules effective in the treatment regimens described herein. For example, in certain embodiments, OSM or a biologically active fragment, mutant, analog or fusion construct thereof is in a glycosylated or pegylated form. Similarly, in certain embodiments, one or more amino acid substitutions or modifications may be introduced into the OSM polypeptide to improve one or more of its properties (e.g., modifications made to render OSM more stable in vivo). In certain embodiments, OSM is human OSM.

In certain aspects, the subject is preferably pre-treated or administered an effective amount of OSM prior to receiving a stem cell transplant. For example, prior to a stem cell transplant, the subject may be administered or pre-treated with an effective amount of OSM about every four, six, eight, twelve, eighteen or twenty-four hours. Similarly, prior to stem cell transplant, the subject may be administered or pre-treated with an effective amount of OSM for about 1, 2, 3, 4, 5, 6, 7, 8, 9, 10, 11, 12, 13, 14, 21, 24 days or longer. In a preferred embodiment, the subject is administered or pre-treated with an effective amount of OSM every six hours for at least two days. In certain embodiments, the subject is administered OSM following the conclusion of the subject's conditioning for engraftment, but in any event prior to stem cell transplant. For example, in some embodiments, OSM is administered to the subject at least one, two, three, four, five, six, seven, ten, twelve, fourteen, twenty one, thirty six, forty two, fifty six, sixty three, seventy, eighty, ninety, one hundred, one hundred and twenty days, six months, nine months, twelve months, or more, after the subject has undergone myeloablative conditioning. Alternatively, in certain embodiments, the subject is administered OSM in conjunction with the subject's conditioning for engraftment (e.g., conditioning of the subject's bone marrow tissues using irradiation).

Stem cell transplant is largely possible because hematopoietic stem cells have an innate ability to home to the bone marrow compartment from the bloodstream after being infused into a subject. The methods and compositions disclosed herein enhance the homing of transplanted stem cells (e.g., HSCs) to one or more target tissues or compartments of the subject. For example, the methods and compositions disclosed herein are useful for enhancing (e.g., increasing) the ability of infused HSCs and/or progenitor stem cells to home to the bone marrow tissue or compartment from the tissues or compartment where such stem cells were infused during transplant (e.g., blood). As used herein, the terms “home” and “homing” mean that the transplanted stem cells (e.g., HSCs or progenitor cells) migrate, move or otherwise concentrate from a first particular tissue, compartment or region where they were infused (e.g., the blood), to a second tissue, compartment or region where they are needed (e.g., the depleted bone marrow compartment of a subject that has undergone conditioning to ablate the endogenous stem cell niche). Various factors within the stem cell niche regulate the homing of HSCs and/or and progenitor stem cells to the bone marrow stem cell niche. One key regulatory mechanism for HSC retention and homing within the bone marrow is expression and production of stromal derived factor-1 (SDF-1)/CXCR4 by stromal cells. Accordingly, in certain aspects disclosed herein, pre-treatment of a subject with OSM stimulates the production of SDF-1 in the bone marrow tissues of the subject.

The reverse of homing is referred to as “mobilization,” which concerns the egress of HSCs and/or progenitor stem cell into the blood stream from the bone marrow stem cell niche and which may be stimulated by G-CSF (filgrastim).

In certain aspects, the compositions and methods disclosed herein increase the homing of transplanted stem cells (e.g., HSCs or progenitor cells) to a target tissue by at least about 10%, 20%, 25%, 30%, 40%, 50%, 60%, 75%, 80%, 90%, 100%, 125%, 150%, 175%, 200%, 250%, 300%, 350%, 400%, 450%, 500%, 600%, 750%, 1,000% or more. In certain aspects, the methods and compositions disclosed herein enhance or increase the homing of engrafted HSCs from the blood, where such HSCs are infused, to a target tissue, such as the bone marrow stem cell niche. In certain embodiments, increasing or enhancing the homing of, for example, transplanted HSCs to the bone marrow stem cell niche improves the likelihood that such transplanted HSCs will engraft and expand within such target tissue and produce mature blood cells.

The present inventors have discovered and describe herein that OSM is a macrophage-produced protein that regulates HSC retention within the stem cell niche. While certain aspects of the present invention contemplate the use or administration of OSM to improve HSC retention or homing to target tissues, the present inventions are not intended to be limited to such embodiments. Rather, also contemplated herein are any methods that may cause an increase in the quantity or production of OSM in a subject or in the target tissues of a subject. For example, macrophage produce positive supporting factors (e.g., OSM) in the target tissue (e.g., the stem cell niche) and such factors are capable of supporting HSC retention. Accordingly, in some aspects, also contemplated are methods of increasing the concentration of macrophage in the target tissues and thereby improving homing to such target tissues. Such an increase in macrophage in the target tissues provide a means of increasing the quantity or production of OSM in the target tissue (e.g., the stem cell niche) and thereby improve homing to and retention of HSCs and/or progenitor stem cells in a subject's target tissues.

In certain aspects, the present inventions relate to methods of increasing a subject's endogenous production of OSM and thereby improving homing to and retention of HSCs and/or progenitor stem cells in a subject's target tissues. For example, contemplated herein are novel expression vectors and methods which facilitate or increase the production of OSM (e.g., SEQ ID NO: 1) or a biologically active fragment, mutant, analog or fusion construct thereof by a cell (e.g., a subject's endogenous cells). In certain aspects, such cells, clones or cell lines demonstrate high OSM expression and secretion efficiency, relative to, for example, an unmodified cell. Such cells that stably express OSM may be administered to a subject in need thereof, thereby improving homing to and retention of HSCs and progenitor stem cell in a subject's target tissues. Similarly, in certain aspects a subject's endogenous cells may be modified such that they express OSM.

Generally, the production of stable mammalian cell lines that efficiently express OSM (e.g., SEQ ID NO: 1) or a biologically active fragment, mutant, analog or fusion construct thereof begins with the transfection of the selected cell line with the gene of interest and usually a selectable marker of gene expression, such that the gene of interest and the selectable marker can be expressed in the host cell line. The gene of interest and the nucleic acids encoding a selectable marker of gene expression can be cloned into, and expressed from a single vector, or alternatively from two separate vectors that are co-transfected.

As such term is used herein, the term “expression vector” refers to a vector, in particular a DNA vector such as a plasmid, which comprises a sequence encoding the gene of interest (e.g., OSM or a biologically active fragment, mutant, analog or fusion construct thereof), and optionally other sequences which are needed or are useful for efficient expression of such gene product. For example, the expression vector of the present invention is one into which a gene encoding OSM or a biologically active fragment, mutant, analog or fusion construct thereof, may be inserted (e.g., by restriction and ligation) such that the gene is operatively linked to one or more regulatory sequences (e.g., nucleic acids encoding a selectable marker of gene expression) and may be expressed by a transfected clone as a gene product. In certain aspects, a transfected clone stably expressing OSM or a biologically active fragment, mutant, analog or fusion construct thereof may be administered to a subject in need thereof (e.g., administered to the target tissues of a subject in need thereof), thereby improving homing to and retention of HSCs in the subject's target tissues.

As used herein, the term “target tissue” generally refers to any tissues or biological compartments of a subject where the engrafted stem cells are needed. For example, in a preferred embodiment, such target tissues comprise the stem cell niche of the bone marrow tissue or compartment. In certain preferred embodiments, the target tissue is or comprises a subject's conditioned or ablated bone marrow stem cell niche. In certain aspects, the target tissues of the subject have been conditioned (e.g., using irradiation or chemotherapy) to ablate an endogenous population of cells from the stem cell niche of the target tissue, such that the subject is ready to accept the transplanted stem cells. For example, in certain embodiments, prior to stem cell transplant the subject's bone marrow tissues have been conditioned to ablate or otherwise deplete the subject's endogenous stem cells from the bone marrow stem cell niche.

Aspects of the methods disclosed herein contemplate conditioning a subject in need of a peripheral blood stem cell transplant for engraftment of transplanted peripheral blood stem cells. As used herein “engrafting” and “engraftment” of a stem cell, including an expanded hematopoietic stem cell, means placing the stem cell into an animal, e.g., by injection, wherein the stem cell persists in vivo. This can be readily measured by the ability of, for example, an engrafted hematopoietic stem cell and/or progenitor stem cell population to contribute to the ongoing blood cell formation in a subject. As used herein, the phrase “conditioning a subject for engraftment” refers to the process of depleting the amount of cells (e.g., hematopoietic stem cells and/or progenitor cells) in a subject's stem cell niche (e.g., bone marrow) for subsequent engraftment in the stem cell niche of healthy transplanted stem cells (e.g., peripheral blood stem cells, bone marrow stem cells, umbilical cord stem cells, genetically modified stem cells, minimally manipulated stem cells, etc.). The disclosure contemplates any conditioning method which would be appropriate in the course of a particular subject's treatment, as well as any stem cell source which would be a desirable source for transplantation.

In certain aspects, upon having homed to a target tissue (e.g., conditioned bone marrow tissue) the transplanted stem cells expand and repopulate such target tissue and subsequently produce mature cells. For example, transplanted HSCs and/or progenitor stem cells may home to the conditioned bone marrow tissue of a subject, expand within such bone marrow tissue to repopulate the depleted stem cell niche and produce mature blood cells to repopulate and restore the hematopoietic system of the subject.

In certain aspects, the compositions and methods disclosed herein also enhance or otherwise increase expansion of the engrafted stem cells (e.g., HSCs and progenitor cells) in the target tissues of the subject. For example, in certain embodiments, administering OSM to a subject prior to stem cell transplant increases the expansion of such transplanted stem cells in the target tissues of the subject. In certain embodiments, pre-treating a subject with OSM prior to stem cell engraftment results in an increase in the expansion of the engrafted stem cells (e.g., HSCs) in the target tissues by at least about 10%, 20%, 25%, 30%, 40%, 50%, 60%, 75%, 80%, 90%, 100%, 125%, 150%, 175%, 200%, 250%, 300%, 350%, 400%, 450%, 500%, 600%, 750%, 1,000% or more.

In some embodiments, following expansion of the engrafted stem cells in the target tissues (e.g., the ablated bone marrow stem cell niche), such stem cells repopulate the target tissues and thereby restore such target tissue. For example, in those embodiments where the target tissue comprises the conditioned bone marrow stem cell niche, transplanted HSCs repopulate the bone marrow stem cell niche, thereby restoring the subject's hematopoietic system (e.g., as evidenced by the subject's production of neutrophils, platelets and white blood cells). In certain aspects, the compositions and methods disclosed herein advantageously improve survival of the subject post-transplant, relative to, for example, methods performed without pre-treating the subject with OSM.

In certain aspects, the compositions and methods disclosed herein are useful for treating or curing a subject affected by a disease (e.g., a stem cell disorder) that may benefit from hematopoietic stem cell or progenitor cell transplantation (e.g., sickle cell disease), including, for example autologous, allogeneic, gene-modified and gene-therapy methods. As used herein, the phrase “stem cell disorder” broadly refers to any disease, disorder or condition that may be treated or cured by conditioning a subject's target tissues for engraftment, and/or by ablating an endogenous stem cell population in a target tissue (e.g., ablating an endogenous HSC or progenitor cell population from a subject's bone marrow tissue) and/or by engrafting or transplanting stem cells in a subject's target tissues. For example, any stem cell disorder that has been shown to be cured by hematopoietic stem cell transplant and may benefit from the methods and compositions of the present inventions. Similarly, in certain aspects, the compositions and methods disclosed herein may be used in connection with conditioning of a subject's bone marrow tissues for engraftment in connection with treatment of such subject's hematological malignancy. In certain aspects, the methods and compositions disclosed herein may be used in connection with stem cell transplant therapy to treat, cure or correct stem cell disorders or diseases selected from the group consisting of the following diseases: sickle cell anemia, thalassemias, Fanconi anemia, Wiskott-Aldrich syndrome, adenosine deaminase SCID (ADA SCID), HIV, metachromatic leukodystrophy, Diamond-Blackfan anemia and Schwachman-Diamond syndrome. In some embodiments, the subject has or is affected by an inherited blood disorder (e.g., sickle cell anemia) or an autoimmune disorder. In some embodiments, the subject has or is affected by a malignancy. For example, a malignancy selected from the group consisting of hematologic cancers (e.g., leukemia, lymphoma, multiple myeloma and other plasma cell dyscrasias (PCD), or myelodysplastic syndrome) and neuroblastoma. In some embodiments, the subject has or is otherwise affected by a metabolic disorder. For example, in certain aspects the subject may suffer or otherwise be affected by a metabolic disorder selected from the group consisting of glycogen storage diseases, mucopolysccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper IGM syndrome, Chédiak-Higashi disease, hereditary lymphohistiocytosis, osteopetrosis, osteogenesis imperfect, the storage diseases, thalassemia major, sickle cell disease, systemic sclerosis, systemic lupus erythematosus, multiple sclerosis, juvenile rheumatoid arthritis and those diseases or disorders described in “Bone Marrow Transplantation for Non-Malignant Disease,” ASH Education Book, 2000 (1) 319-338, the contents of which are incorporated herein by reference in their entirety.

As used herein, the terms “treat,” “treatment,” “treating,” or “amelioration” when used in reference to a disease, stem cell disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of, for example, a condition, disease, or disorder described herein, or delaying or slowing onset of a condition, disease, or disorder described herein, and an increased lifespan as compared to that expected in the absence of treatment.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient,” “individual” and “subject” are used interchangeably herein. Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, of a hematological malignancy. In addition, the methods described herein can be used to treat domesticated animals and/or pets. A subject can be male or female.

A subject can be one who has been previously diagnosed with or identified as suffering from or having a condition, disease, or stem cell disorder described herein and in need of treatment (e.g., of a hematological malignancy or non-malignant disease described herein) or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. Rather, a subject can include one who exhibits one or more risk factors for a condition or one or more complications related to a condition. A “subject in need” of treatment for a particular condition can be a subject having that condition, diagnosed as having that condition, or at increased risk of developing that condition relative to a given reference population.

In some embodiments, the methods described herein comprise selecting a subject diagnosed with, suspected of having, or at risk of developing a hematological malignancy, for example a hematological malignancy described herein.

In some embodiments, the methods described herein comprise selecting a subject diagnosed with, suspected of having, or at risk of developing a non-malignant disease, for example a non-malignant disease described herein.

Oncostatin M and any biologically active fragment, mutant, analog or fusion construct thereof may be formulated for administration to a subject (e.g., a mammal) using any suitable routes of administration (e.g., orally, parenterally, intramuscularly, subcutaneously, topically, nasally or transdermally). Accordingly, also disclosed herein are pharmaceutical compositions that comprise OSM or a biologically active fragment, mutant, analog or fusion construct thereof. OSM can be formulated in pharmaceutically acceptable compositions which comprise a therapeutically-effective amount of OSM, formulated together with one or more pharmaceutically acceptable carriers (additives) and/or diluents. The formulations can conveniently be presented in unit dosage form and may be prepared by any of the methods well known in the art of pharmacy. Techniques, excipients and formulations generally are found in, e.g., Remington's Pharmaceutical Sciences, Mack Publishing Co., Easton, Pa. 1985, 17th edition, Nema et al., PDA J. Pharm. Sci. Tech. 1997 51:166-171; and, L. William, Remington: The Science and Practice of Pharmacy. 22nd ed. Pharmaceutical Press (2012), the entire contents of which are incorporated herein by reference.

In certain aspects, OSM can be administered to a subject in combination with other pharmaceutically active agents. Exemplary pharmaceutically active agents include, but are not limited to, those found in Harrison's Principles of Internal Medicine, 13th Edition, Eds. T. R. Harrison et al. McGraw-Hill N.Y., NY; Physician's Desk Reference, 50th Edition, 1997, Oradell New Jersey, Medical Economics Co.; Pharmacological Basis of Therapeutics, 8th Edition, Goodman and Gilman, 1990; United States Pharmacopeia, The National Formulary, USP XII NF XVII, 1990, the complete contents of all of which are incorporated herein by reference. In some embodiments, the pharmaceutically active agent is a conventional treatment for a hematological malignancy. In some embodiments, the pharmaceutically active agent is a conventional treatment for a non-malignant disease. The skilled artisan will be able to select the appropriate conventional pharmaceutically active agent for treating any particular hematological malignancy or non-malignant disease using the references mentioned above based on their expertise, knowledge and experience.

In some embodiments, the compositions of the present invention may be packaged as a kit, which may contain one or more unit dosage forms containing OSM or a biologically active fragment, mutant, analog or fusion construct thereof. Such a kit may optionally be accompanied by instructions for use or administration. As used herein, the term “effective amount” means an amount sufficient to achieve a meaningful benefit to the subject. For example, an effective amount of the compositions that are the subject of the present inventions may be generally determined based on the activity of OSM and the amount of OSM necessary to enhance homing of transplanted stem cells to a target tissue and the subsequent engraftment of such stem cells in such target tissue. In certain aspects, an effective amount of the compositions disclosed herein refers to an amount of an active ingredient (e.g., OSM or a biologically active fragment or analog thereof) that is effective to increase or enhance homing or retention of HCSs to the target tissues (e.g., the stem cell niche) of a subject. An effective amount of such compositions can be readily determined depending on the subject's disease and other related characteristics. Such characteristics include the condition, general health, age, subjective symptoms, objective appearance, sex and body weight of the subject.

In some contexts, administration of the OSM-containing compositions described herein increase engraftment of transplanted stem cells to the target tissues of the subject. The terms “increased”, “increase” or “enhance” are all used herein to generally mean an increase by a statically significant amount; for the avoidance of any doubt, the terms “increased”, “increase” or “enhance” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In some embodiments, administration the OSM-containing compositions disclosed herein increases engraftment efficiency or retention of transplanted hematopoietic stem cells and/or progenitor cells in the target tissue by at least about 5%, 10%, 20%, 30%, 40%, 50%, 60%, 70%, 80, 90%, or as much as 100% as compared to a reference level. In some embodiments, administration the OSM-containing compositions disclosed herein increases hematopoietic stem cells and/or progenitor cells engraftment in the target tissue by at least about 1.1-fold, 1.2-fold, 1.3-fold, 1.4-fold, 1.5-fold, 2-fold, 3-fold, 4-fold, 5-fold, or at least about a 10-fold or greater as compared to a reference level.

It is to be understood that the invention is not limited in its application to the details set forth in the description or as exemplified. The invention encompasses other embodiments and is capable of being practiced or carried out in various ways. Also, it is to be understood that the phraseology and terminology employed herein is for the purpose of description and should not be regarded as limiting.

While certain agents, compounds, compositions and methods of the present invention have been described with specificity in accordance with certain embodiments, the following examples serve only to illustrate the methods and compositions of the invention and are not intended to limit the same.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications and other reference materials referenced herein to describe the background of the invention and to provide additional detail regarding its practice are hereby incorporated by reference.

### EXAMPLES

### Example 1

Recently, three independent groups (Winkler, et al., Blood 2010; 116(23):4815-28; Christopher, et al., Journal of Experimental Medicine 2011; 208(2): 251-260; and Chow, et al., Journal of Experimental Medicine 2011; 208(2): 261-271) have implicated macrophages as a key regulator in granulocyte-colony stimulating factor (G-CSF)-mediated mobilization of HSCs. These groups have demonstrated that macrophages express the G-CSF receptor, and that depletion of macrophages leads to niche attenuation, reduced levels of stromal derived factor-1 (SDF-1), and mobilization of HSCs. This data implied that macrophages produce a positive supporting factor in the niche that supports HSC retention, though none of these prior reports were able to identify the key factors.

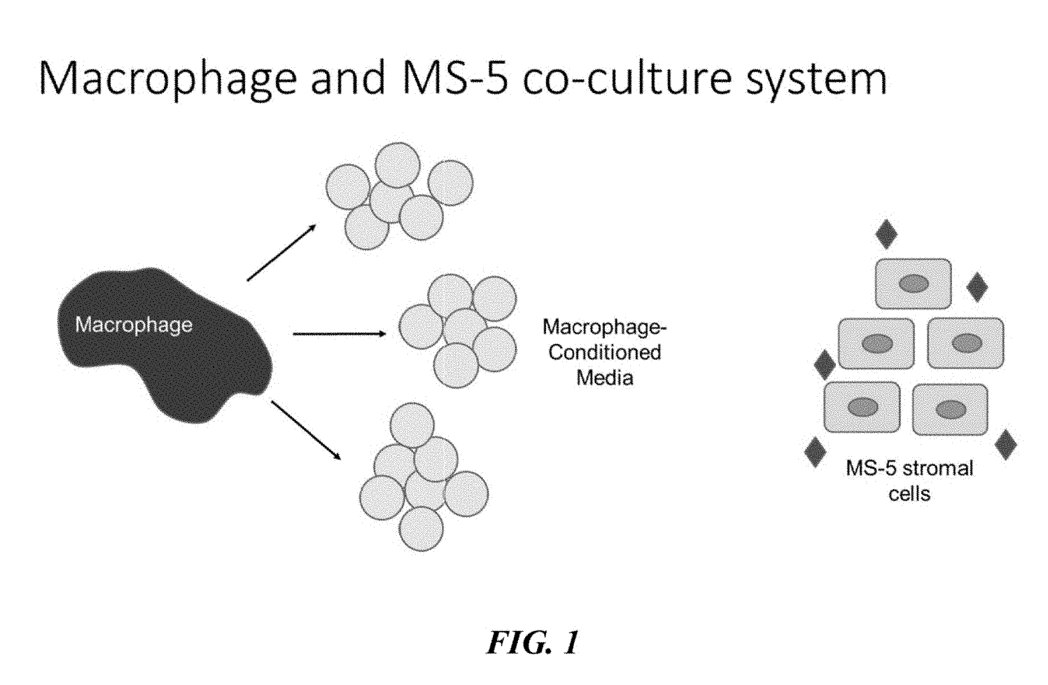

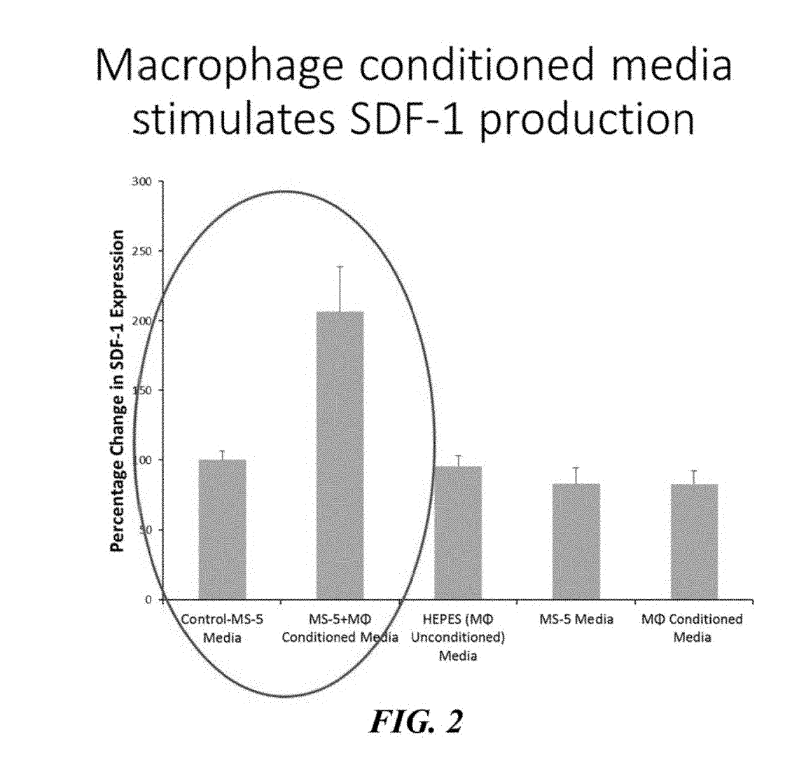

The present inventors have discovered and describe herein that oncostatin M (OSM) is a macrophage-produced protein that regulates hematopoietic stem cell (HSC) retention within the stem cell niche. In an effort to determine what factors macrophages might be secreting that supports the niche, the present inventors began by setting up a co-culture system in which media containing secretions from macrophages was collected and then cultured together with SDF-1 producing stromal cells, as depicted in FIG. 1. Initial identification was achieved utilizing such in vitro co-culture screening system and, as depicted in FIG. 2, the present inventors confirmed that macrophages stimulated SDF-1 production, and then moved to screen for possible candidate factors responsible for this observed stimulation. In contrast, when OSM production was reduced via shRNA knockdown, or the OSM receptor was knocked down in MS-5 cells or blocked with antibody, the enhanced SDF-1 production was abrogated. Intriguingly, this effect was specific to OSM, as changes in IL-6 or LIF signaling, both members of a similar gp130 signaling family along with OSM, did not have any effects.

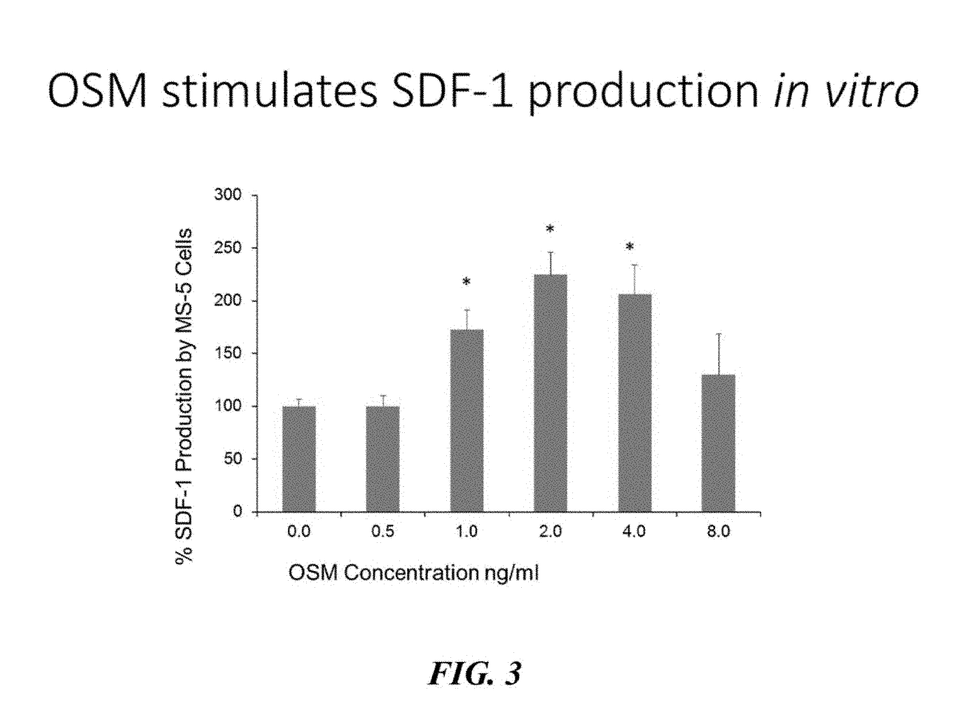

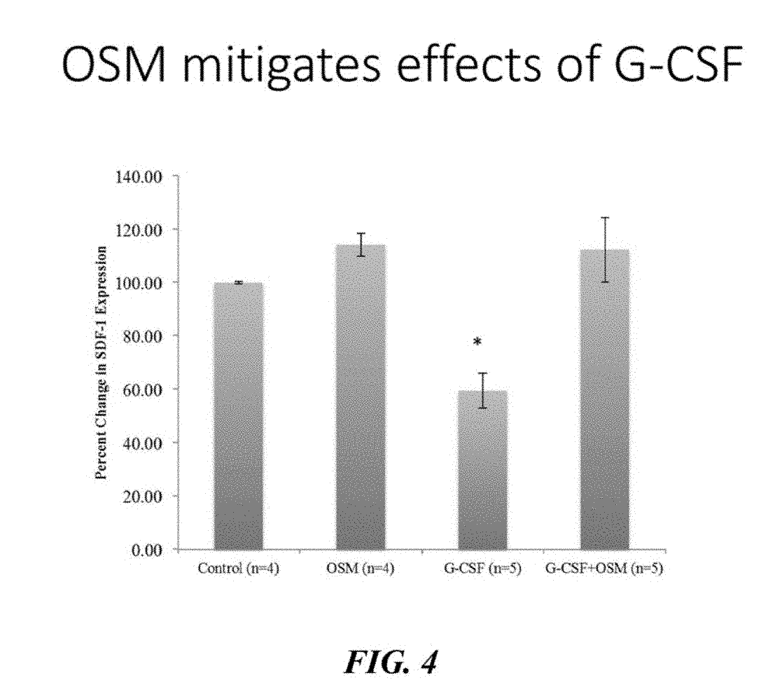

In screening for possible candidate factors responsible for the observed stimulation of SDF-1 production, the present inventors determined that the lead candidate molecule was OSM, which, as shown in FIG. 3, also stimulates SDF-1 production when cultured with stromal cells in vitro. As illustrated in FIG. 4, not only did OSM stimulate production of SDF-1, but it also mitigated the negative regulatory effects of G-CSF on SDF-1 production, signaling that OSM could potentially reverse HSC mobilization in vivo and suggesting that OSM was the key factor secreted by macrophages to regulate HSC retention within the niche.

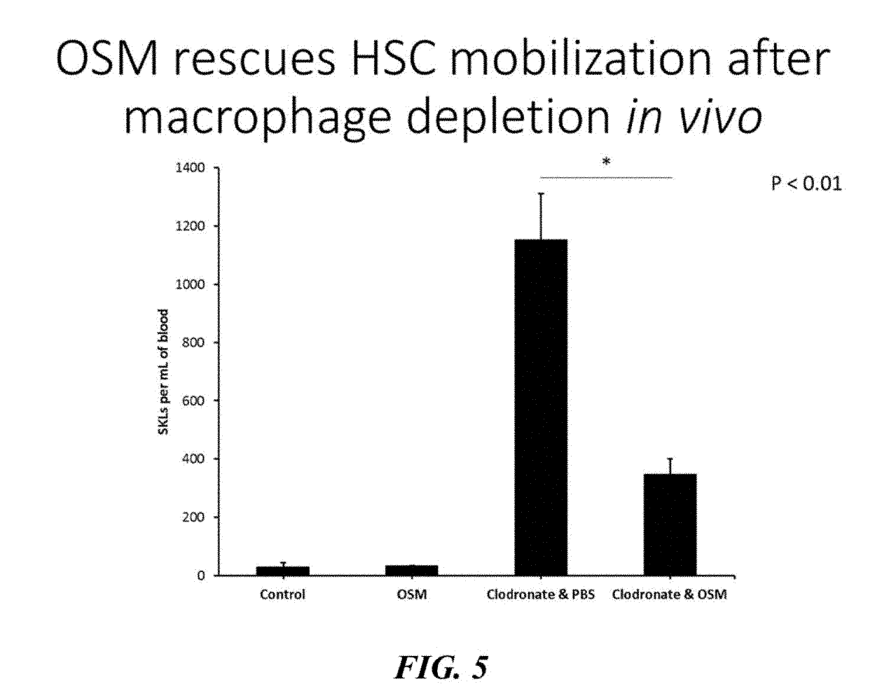

When mice were treated G-CSF, significant reductions in bone marrow SDF-1 levels and mobilization resulted. However, when mice were co-treated with OSM, levels of bone marrow SDF-1 remained the same as untreated mice, as illustrated in FIG. 5 (n=10 mice per group, two independent experiments, P<0.01). Similarly, as illustrated in FIG. 5, when macrophages were depleted in vivo via clodronate-loaded liposome treatment, significant HSC mobilization occurred, which was blocked with co-treatment of OSM (n=10 mice per group, 2 independent experiments, P<0.01).

The foregoing results elucidate a previously unknown key regulatory mechanism governing G-CSF and macrophage-mediated mobilization of HSCs.

### Example 2

The present inventors hypothesized that the foregoing results could be leveraged as a novel therapeutic strategy in an effort to enhance HSC homing and engraftment. In particular, the present inventors sought to build on their understanding of the mechanisms governing mobilization of HSCs and to determine whether the ability of OSM to regulate HSC retention could be leveraged as a novel therapeutic for enhancing bone marrow transplantation.

When HSCs are transplanted they must, (1) traffic from the blood where they are infused and “home” to the bone marrow niche, (2) expand within the bone marrow to repopulate the conditioned stem cell niche, (3) produce mature blood cells to repopulate the hematopoietic system of the recipient, and (4) keep the recipient subject alive by restoring the hematopoietic system.

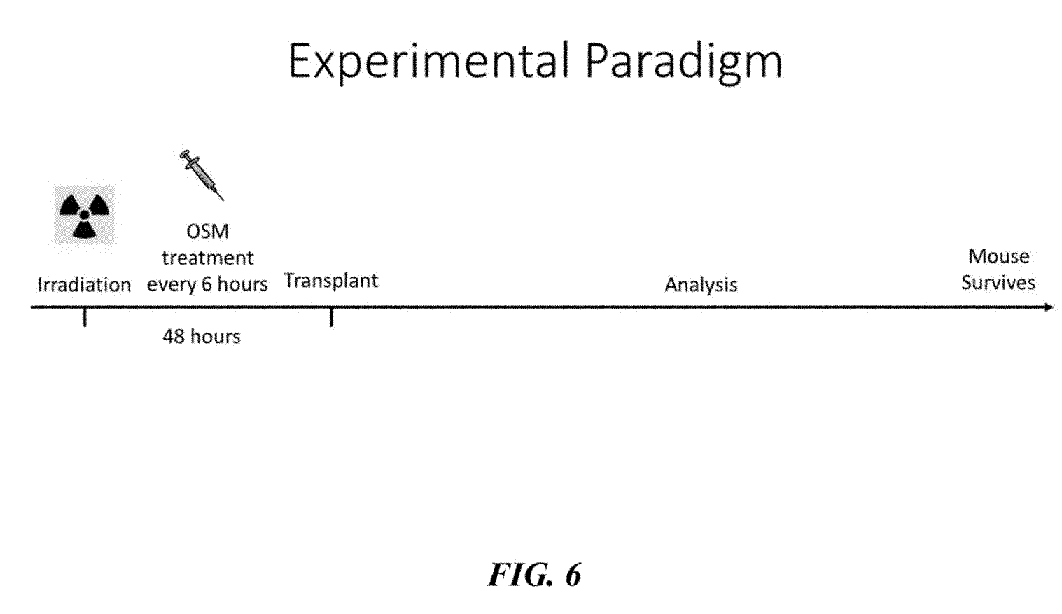

To test the effect of OSM at each of the foregoing stages, the present inventors used the general experimental protocol depicted in FIG. 6 to mimic what happens in a clinical setting. As illustrated in FIG. 6, first the mice were irradiated to mimic the treatment a subject might receive to deplete their blood system. After 48 hours, during which mice received OSM treatment every 6 hours, mice received a transplant, and were then subsequently evaluated.

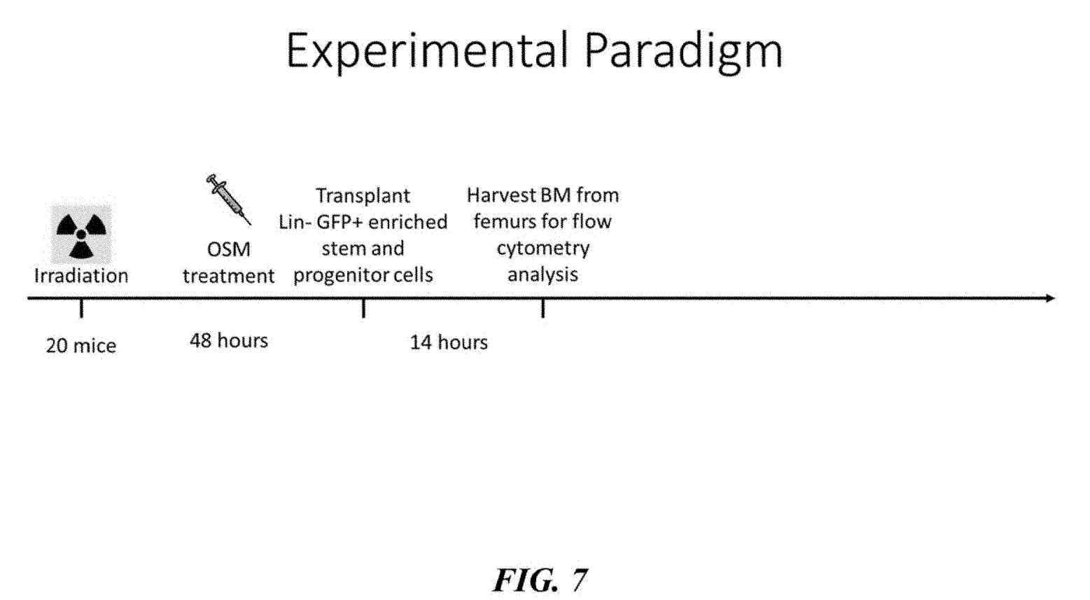

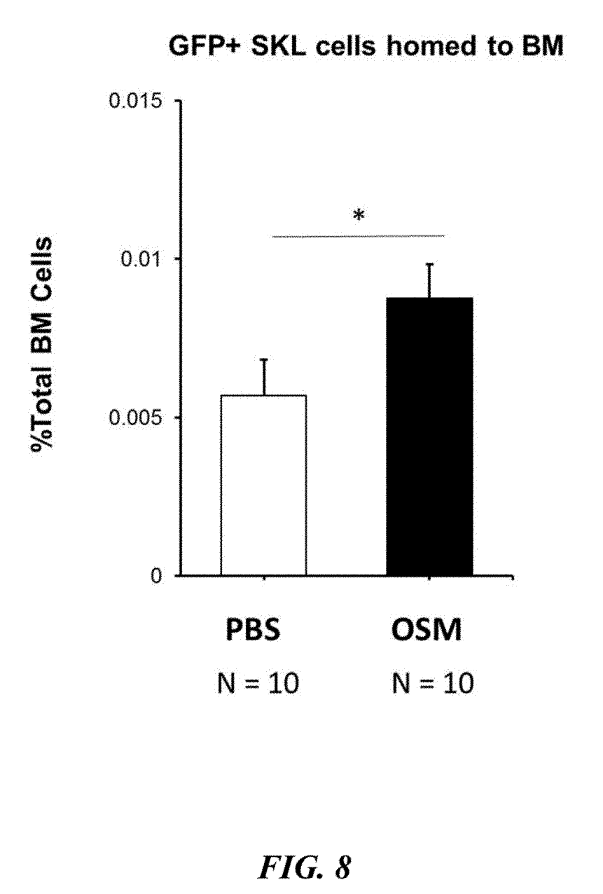

The present inventors first sought to determine whether OSM treatment could improve homing of HSCs to the bone marrow tissue by conducting the experimental protocol depicted in FIG. 7. As illustrated in FIG. 7, the experimental protocol for this study was as follows: 20 mice irradiated, treated with recombinant OSM (0.5 μg per injection) every 6 hours for 48 hours or vehicle control, and then transplanted with an enriched population of GFP⁺ stem and progenitor cells; 14 hours after transplant mice were analyzed, femurs were pulled and flushed to obtain bone marrow for flow cytometry analysis looking at how many GFP⁺ cells and GFP⁺ HSCs from the donor were present or “homed” in the bone marrow. As illustrated in FIG. 8, mice pre-treated with OSM prior to transplantation exhibited a 2-fold increase in HSC homing compared to vehicle control groups (n=10 mice per group, three independent experiments, P<0.05).

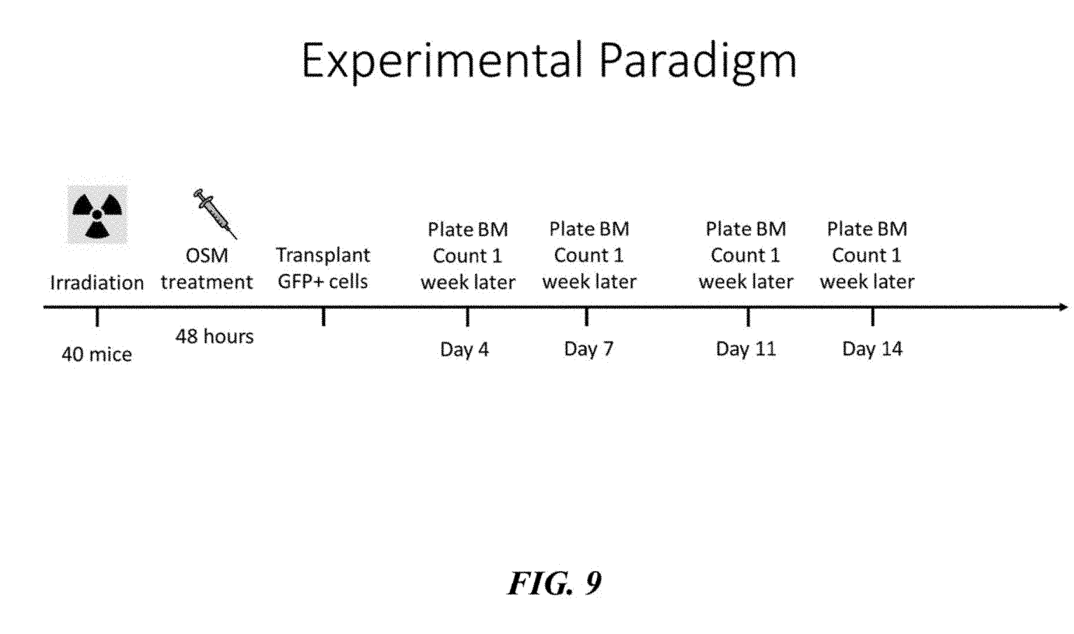

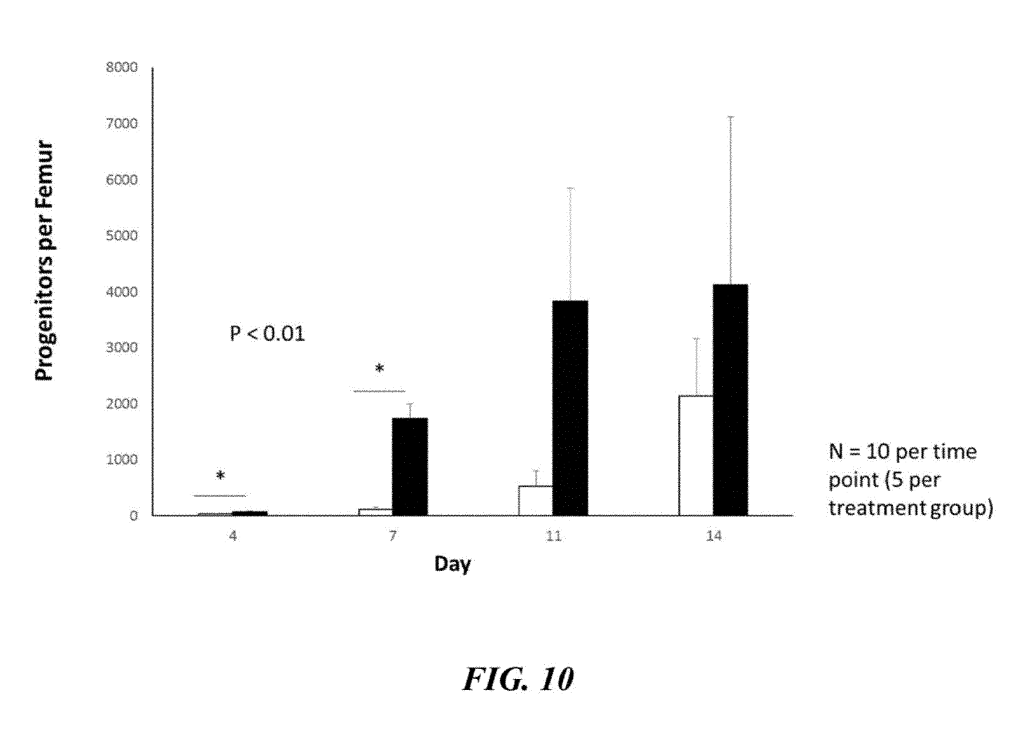

Next, to assess whether these homed cells were able to expand and repopulate the niche, the present inventors followed the experimental protocol depicted in FIG. 9. Cohorts of mice were sacrificed at days 4, 7, 11, and 14, bone marrow was plated in methylcellulose, and the number of colony forming units (CFUs) were counted as a measure of progenitor and stem cell expansion. As illustrated in FIG. 10, these homed progenitors demonstrated remarkably enhanced hematopoietic expansion in OSM treated mice, as demonstrated by increased numbers of CFUs from bone marrow assessed days 4, 7, 11, and 14 post-transplant (n=10 mice per group, P<0.01).

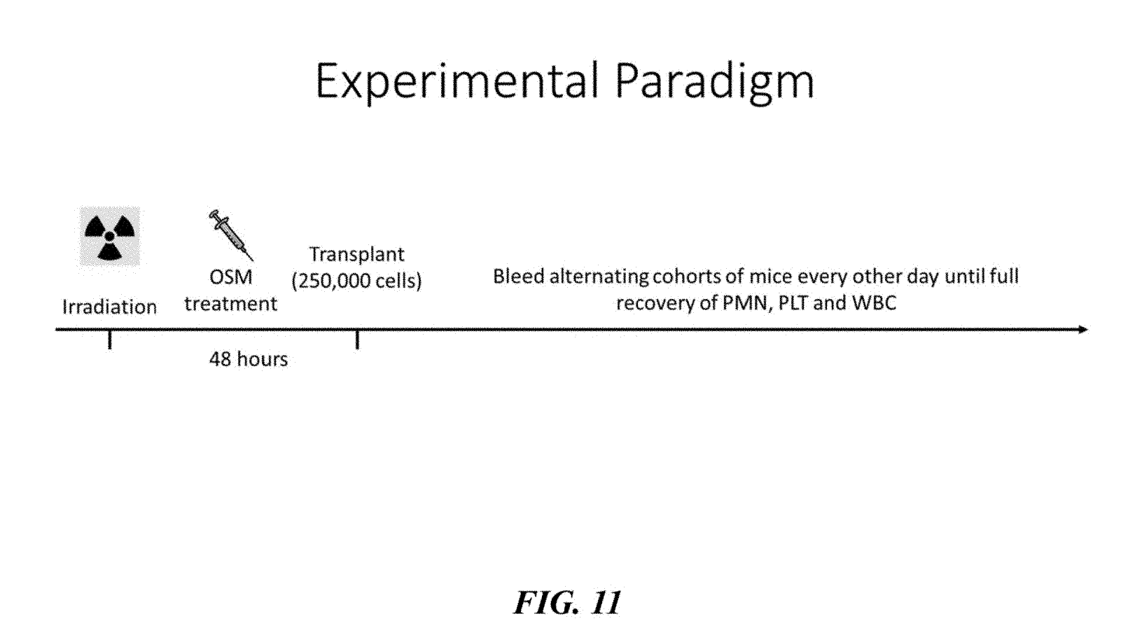

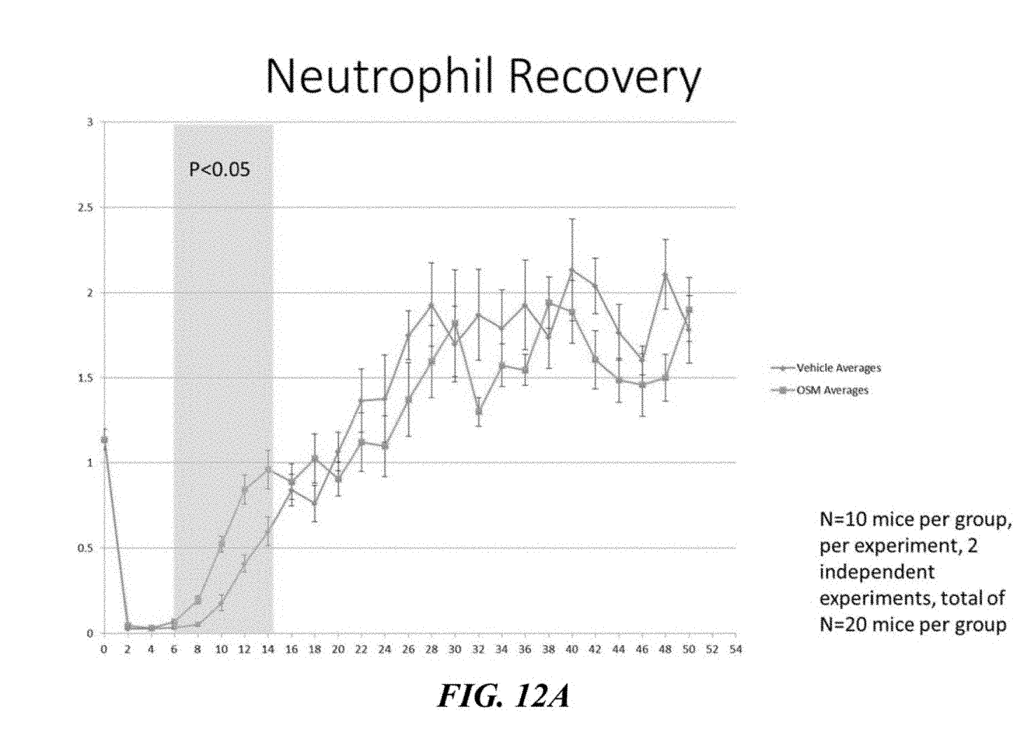

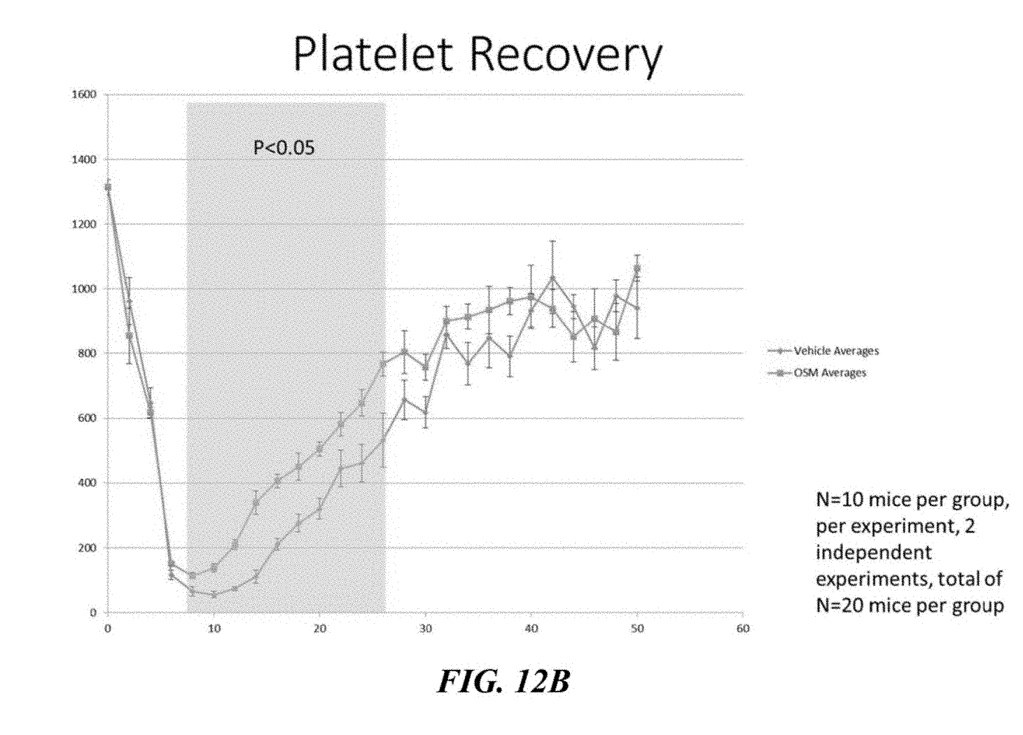

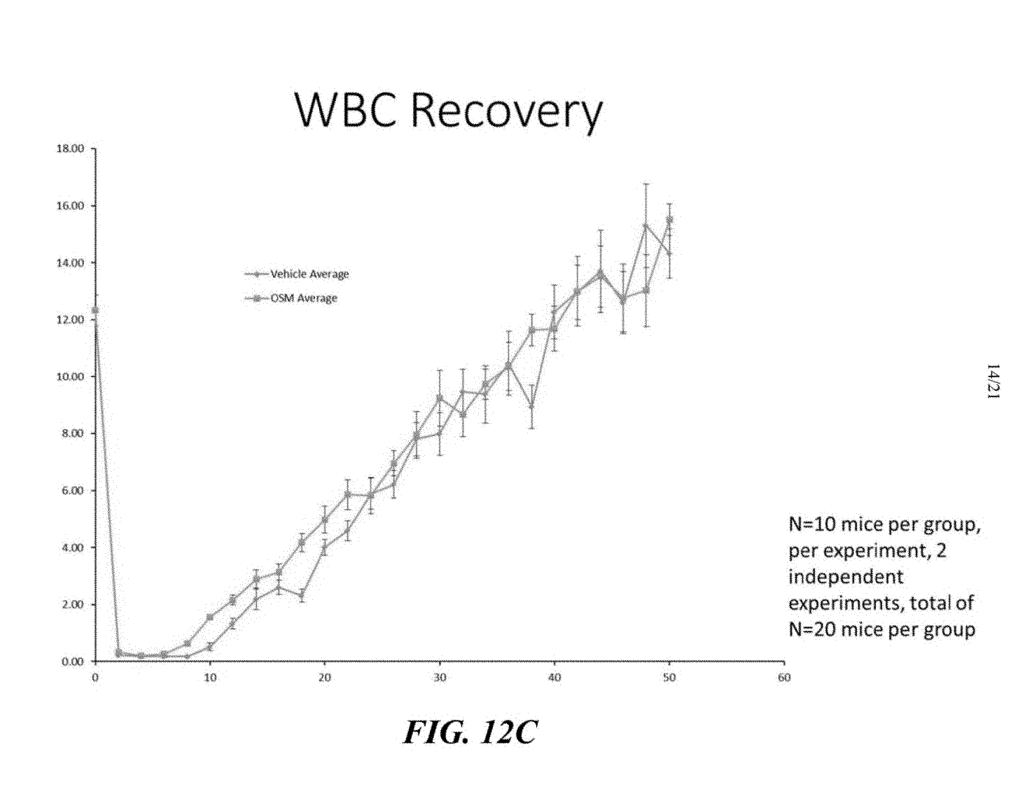

The present inventors next conducted the experimental protocol illustrated in FIG. 11 to determine whether transplanted HSCs produce mature blood cells to repopulate the hematopoietic system of the recipient. As illustrated in FIGS. 12A-C respectively, the average neutrophil, platelet and white blood cell recoveries were comparable, and in many instances higher, in the OSM pre-treated mice, thereby confirming that the transplanted HSCs produce mature blood cells to repopulate the hematopoietic system of the recipient mice.

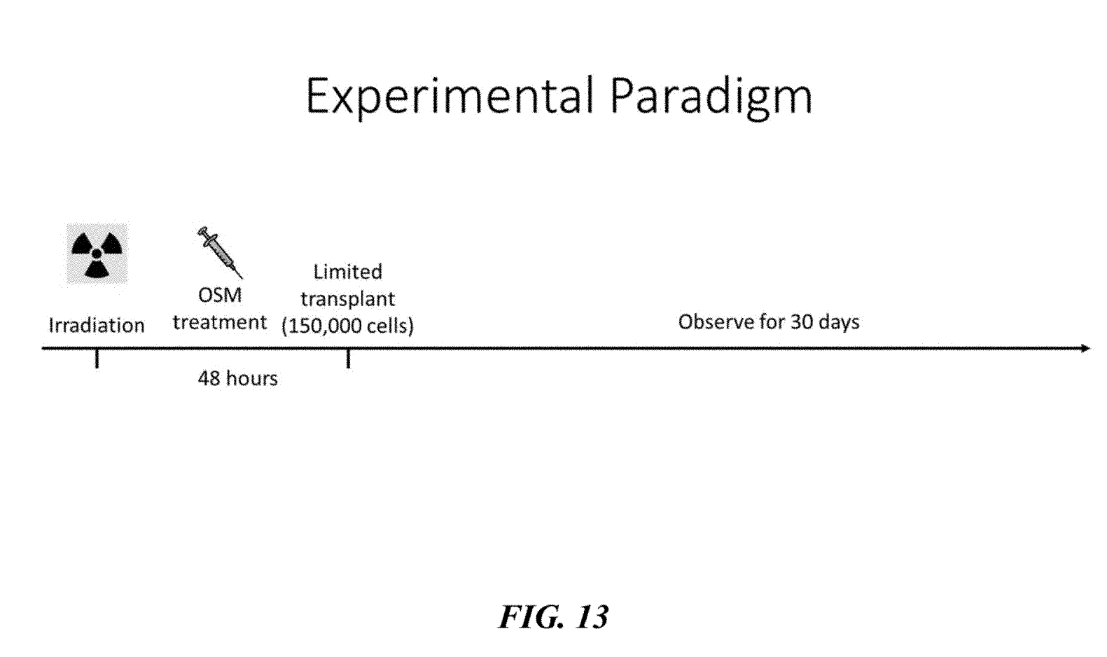

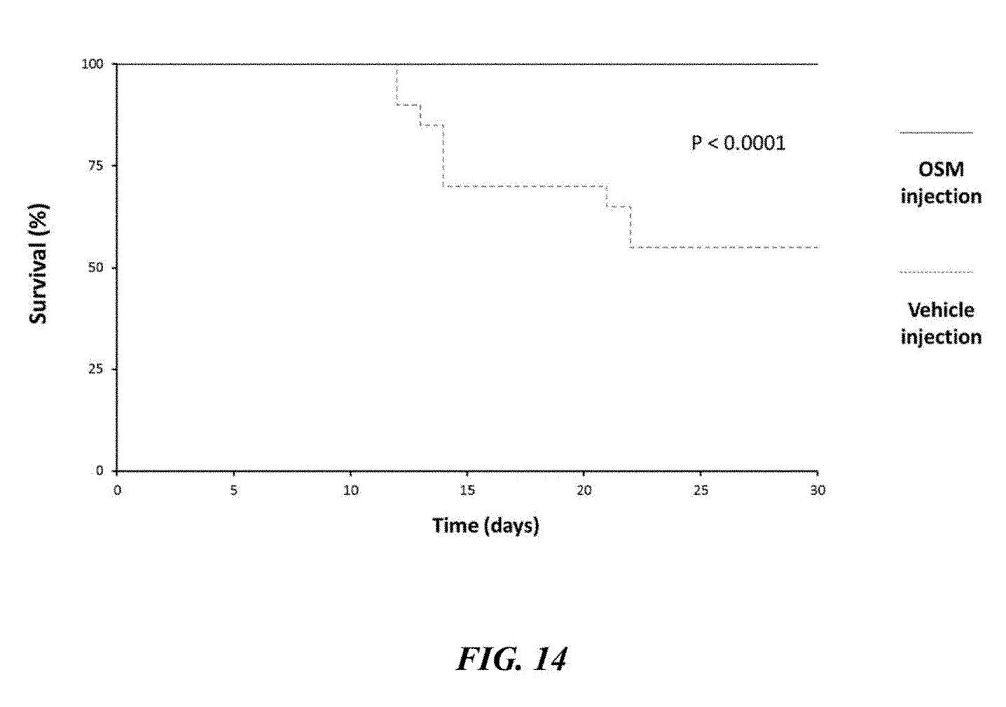

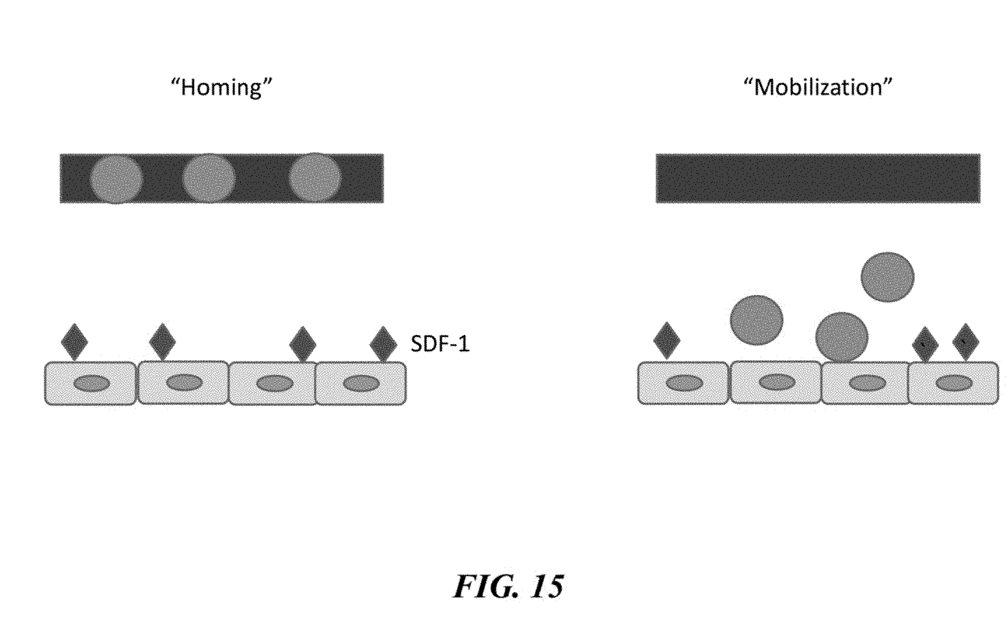

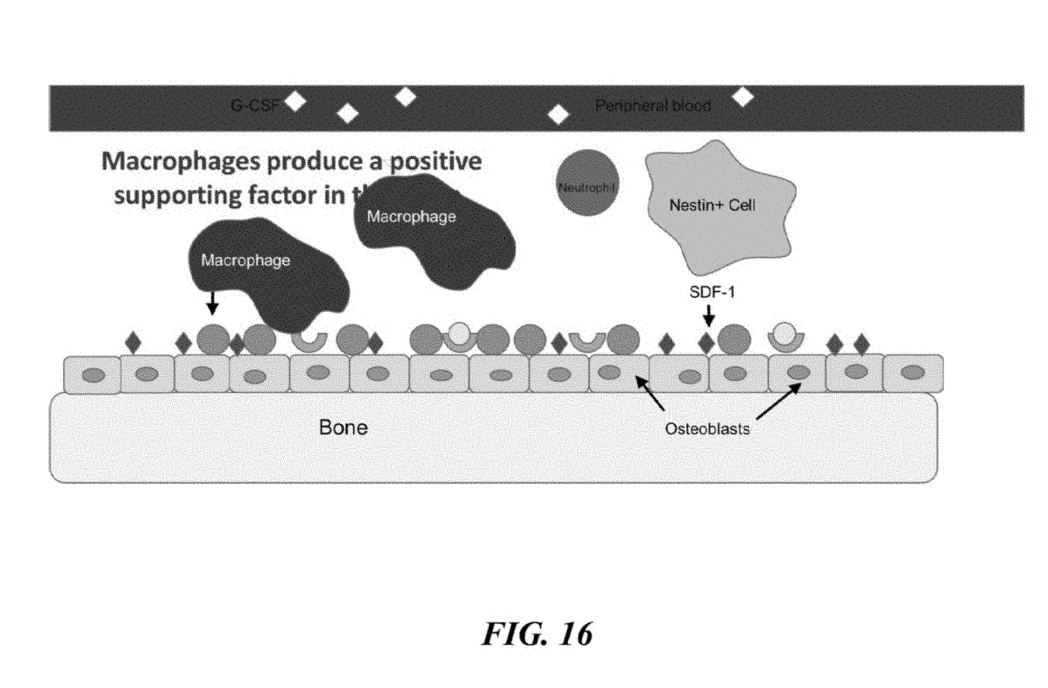

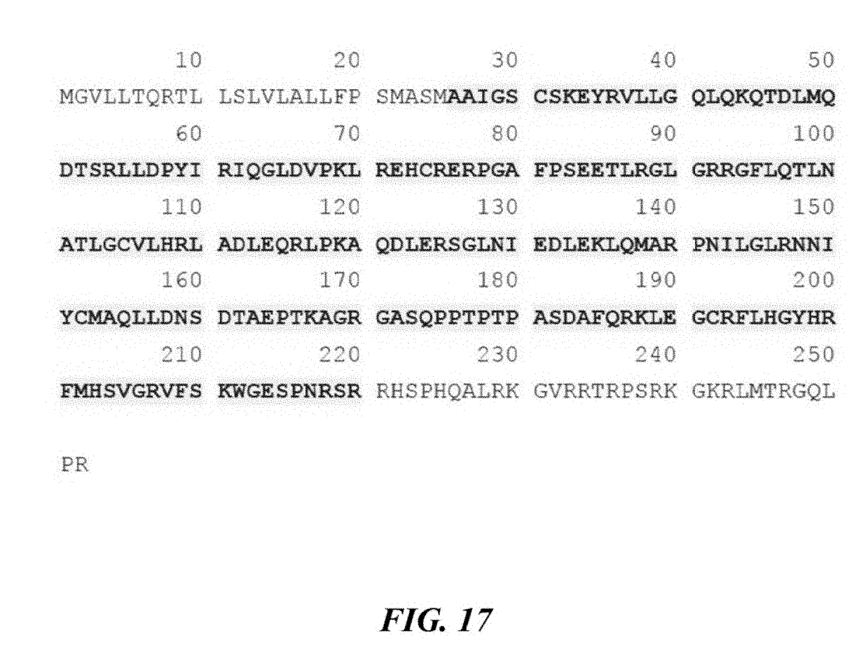

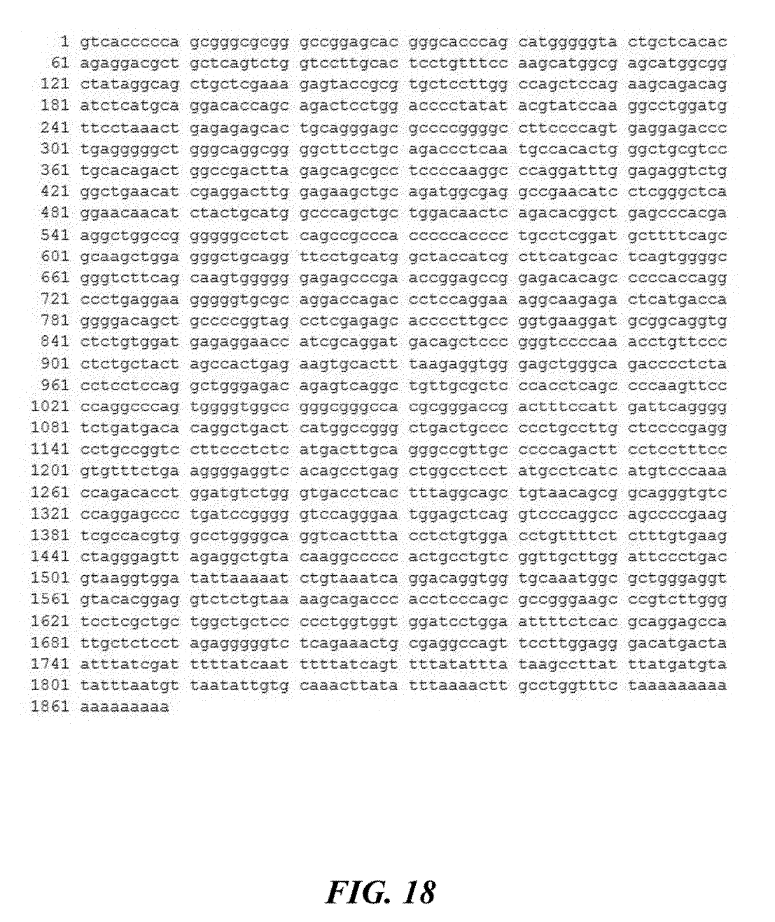

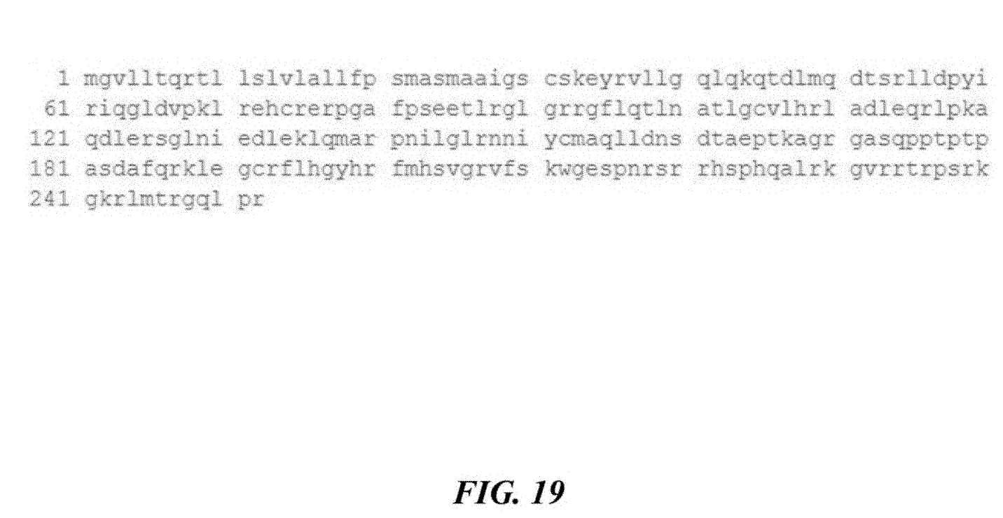

The present inventors next sought to determine whether the observed expansion and repopulation leads to increased survival after transplant. To study whether the improved homing and expansion observed after OSM pre-treatment translates to improved survival, the present inventors followed the same experimental protocol depicted in FIG. 13 and, after transplant, mice were observed for 30 days to determine the percentage that survived the procedure. As illustrated in FIG. 14, using a limited cell number transplant mimicking settings of single cord blood unit transplantation in adults or other settings of limited HSC number, the present inventors demonstrated enhanced hematopoietic engraftment and survival in OSM pre-treated mice, with 100% survival compared to 50% survival of mice treated with vehicle control (n=20 per group, P<0.001).

Collectively, the results presented herein demonstrate how illumination of endogenous regulatory mechanisms within the hematopoietic niche can reveal molecular agents and pathways with potential to serve as new therapeutic agents in the clinic. Specifically, the present inventors identify herein that OSM is a key regulatory factor governing the G-CSF-macrophage-mediated mobilization of HSCs, and describe for the first time a novel therapeutic approach using recombinant OSM as a therapeutic to meet a currently unmet clinical need for HSC transplantation.

The foregoing results thus evidence that pre-treatment with OSM enhances stem and progenitor homing to the bone marrow, expansion and repopulation of cells within the bone marrow and survival post-transplant, thereby evidencing that OSM can serve as a potential new therapeutic to enhance bone marrow transplant. This could help to meet a currently unmet clinical need, since, as there are no therapies available today to enhance these stages of bone marrow transplant.

1-72. (canceled)

73. A method of increasing stem cell engraftment efficiency, stem cell homing, or stem cell retention in a target tissue of a subject, the method comprising administering oncostatin M or a biologically active fragment, mutant, analog or fusion construct thereof to the subject and thereby increasing the stem cell engraftment efficiency, homing, or retention in the target tissue of the subject.

74. The method of claim 73, wherein the stem cells are administered to the subject.

75. The method of claim 74, wherein the subject is pre-treated with the oncostatin M or a biologically active fragment, mutant, analog or fusion construct thereof prior to administering stem cells to the subject.

76. The method of claim 75, wherein the oncostatin M or a biologically active fragment, mutant, analog or fusion construct thereof is administered to the subject for at least two days prior to administering stem cells to the subject.

77. The method of claim 73, wherein the target tissue comprises bone marrow tissue or a stem cell niche.

78. The method of claim 73, wherein the stem cells are selected from the group consisting of hematopoietic stem cells (HSCs), progenitor cells, hematopoietic progenitor cells, exogenous stem cells, endogenous stem cells, and genetically modified endogenous stem cells.

79. The method of claim 73, wherein the subject receives myeloablative conditioning prior to the step of administering oncostatin M or a biologically active fragment, mutant, analog or fusion construct thereof to the subject.

80. The method of claim 73, wherein the method increases the rate of subject survival as compared to a method performed without the step of administering oncostatin M or a biologically active fragment, mutant, analog or fusion construct thereof to the subject.

81. The method of claim 73, wherein the subject has or is affected by a hematologic or oncologic disease.

82. The method of claim 81, wherein the subject has or is affected by a hematologic or oncologic disease selected from the group consisting of leukemia, lymphoma, multiple myeloma and myelodysplastic syndrome.

83. The method of claim 73, wherein the subject has or is affected by a hematological malignancy.

84. The method of claim 83, wherein the hematological malignancy is selected from the group consisting of acute lymphoid leukemia, acute myeloid leukemia, chronic lymphoid leukemia, chronic myeloid leukemia, Hodgkin's disease (HD), diffuse large B-cell non-Hodgkin's lymphoma, mantle cell lymphoma, lymphoblastic lymphoma, Burkitt's lymphoma, follicular B-cell non-Hodgkin's lymphoma, T-cell non-Hodgkin's lymphoma, lymphocyte predominant nodular Hodgkin's lymphoma, multiple myeloma, and juvenile myelomonocytic leukemia.

85. The method of claim 73, wherein the subject has or is affected by a non-malignant disease.

86. The method of claim 85, wherein the non-malignant disease is selected from the group consisting of myelofibrosis, myelodysplastic syndrome, amyloidosis, severe aplastic anemia, paroxysmal nocturnal hemoglobinuria, immune cytopenias, systemic sclerosis, rheumatoid arthritis, multiple sclerosis, systemic lupus erythematosus, Crohn's disease, chronic inflammatory demyelinating polyradiculoneuropathy, human immunodeficiency virus (HIV), Fanconi anemia, sickle cell disease, beta thalassemia major, Hurler's syndrome (MPS-IH), adrenoleukodystrophy, metachromatic leukodystrophy, familial erythrophagocytic lymphohistiocytosis and other histiocytic disorders, severe combined immunodeficiency (SCID), and Wiskott-Aldrich syndrome.

87. The method of claim 73, wherein the oncostatin M is human mature oncostatin M or a biologically active fragment or variant thereof.

88. The method of claim 73, wherein the oncostatin M comprises residues 26-220, residues 1-220, or residues 220-252 of SEQ ID NO: 1, or is a biologically active fragment or variant of residues 26-220, residues 1-220, or residues 220-252 of SEQ ID NO: 1.

89. The method of claim 73, wherein the oncostatin M is encoded by SEQ ID NO: 2.

90. The method of claim 73, wherein the oncostatin M comprises SEQ ID NO: 3 or a biologically active fragment or variant thereof.

91. A method of reducing mobilization of stem cells from a target tissue of a subject, the method comprising administering oncostatin M or a biologically active fragment, mutant, analog or fusion construct thereof to the subject and thereby reducing mobilization of the stem cells from the target tissue of the subject.

92. A method of engrafting stem cells in a target tissue of a subject, the method comprising: (a) administering oncostatin M or a biologically active fragment, mutant, analog or fusion construct thereof to the subject; and (b) administering the stem cells to the subject, thereby engrafting the stem cells in the target tissue of the subject.


SEQUENCE LISTING (3 sequences)

SEQ ID NO: 1 - protein, 252 aa, Homo sapiens
MGVLLTQRTLLSLVLALLFPSMASMAAIGSCSKEYRVLLGQLQKQTDLMQDTSRLLDPYIRIQGLDVPKLREHCRERPGAFPSEETLRGLGRRGFLQTLNATLGCVLHRLADLEQRLPKAQDLERSGLNIEDLEKLQMARPNILGLRNNIYCMAQLLDNSDTAEPTKAGRGASQPPTPTPASDAFQRKLEGCRFLHGYHRFMHSVGRVFSKWGESPNRSRRHSPHQALRKGVRRTRPSRKGKRLMTRGQLPR

SEQ ID NO: 2 - DNA, 1869 bp, Homo sapiens
gtcacccccagcgggcgcgggccggagcacgggcacccagcatgggggtactgctcacacagaggacgctgctcagtctggtccttgcactcctgtttccaagcatggcgagcatggcggctataggcagctgctcgaaagagtaccgcgtgctccttggccagctccagaagcagacagatctcatgcaggacaccagcagactcctggacccctatatacgtatccaaggcctggatgttcctaaactgagagagcactgcagggagcgccccggggccttccccagtgaggagaccctgagggggctgggcaggcggggcttcctgcagaccctcaatgccacactgggctgcgtcctgcacagactggccgacttagagcagcgcctccccaaggcccaggatttggagaggtctgggctgaacatcgaggacttggagaagctgcagatggcgaggccgaacatcctcgggctcaggaacaacatctactgcatggcccagctgctggacaactcagacacggctgagcccacgaaggctggccggggggcctctcagccgcccacccccacccctgcctcggatgcttttcagcgcaagctggagggctgcaggttcctgcatggctaccatcgcttcatgcactcagtggggcgggtcttcagcaagtggggggagagcccgaaccggagccggagacacagcccccaccaggccctgaggaagggggtgcgcaggaccagaccctccaggaaaggcaagagactcatgaccaggggacagctgccccggtagcctcgagagcaccccttgccggtgaaggatgcggcaggtgctctgtggatgagaggaaccatcgcaggatgacagctcccgggtccccaaacctgttcccctctgctactagccactgagaagtgcactttaagaggtgggagctgggcagacccctctacctcctccaggctgggagacagagtcaggctgttgcgctcccacctcagccccaagttccccaggcccagtggggtggccgggcgggccacgcgggaccgactttccattgattcaggggtctgatgacacaggctgactcatggccgggctgactgcccccctgccttgctccccgaggcctgccggtccttccctctcatgacttgcagggccgttgcccccagacttcctcctttccgtgtttctgaaggggaggtcacagcctgagctggcctcctatgcctcatcatgtcccaaaccagacacctggatgtctgggtgacctcactttaggcagctgtaacagcggcagggtgtcccaggagccctgatccgggggtccagggaatggagctcaggtcccaggccagccccgaagtcgccacgtggcctggggcaggtcactttacctctgtggacctgttttctctttgtgaagctagggagttagaggctgtacaaggcccccactgcctgtcggttgcttggattccctgacgtaaggtggatattaaaaatctgtaaatcaggacaggtggtgcaaatggcgctgggaggtgtacacggaggtctctgtaaaagcagacccacctcccagcgccgggaagcccgtcttgggtcctcgctgctggctgctccccctggtggtggatcctggaattttctcacgcaggagccattgctctcctagagggggtctcagaaactgcgaggccagttccttggagggacatgactaatttatcgatttttatcaatttttatcagttttatatttataagccttatttatgatgtatatttaatgttaatattgtgcaaacttatatttaaaacttgcctggtttctaaaaaaaaaaaaaaaaaa

SEQ ID NO: 3 - protein, 252 aa, Homo sapiens; identical to SEQ ID NO: 1